(19) United States
(12) Patent Application Publication
(10) Pub. No.: US 2024/0398086 A1
(43) Pub. Date: Dec. 5, 2024

(54) HAIR CARE DEVICE

(71) Applicant: Panasonic Intellectual Property Management Co., Ltd., Osaka (JP)

(72) Inventors: Aya ISHIHARA, Osaka (JP); Miei KINOSHITA, Shiga (JP); Hiroyuki INOUE, Shiga (JP); Yuki CHIKAZAWA, Nara (JP)

(21) Appl. No.: 18/259,715

(22) PCT Filed: Dec. 24, 2021
(86) PCT No.: PCT/JP2021/048153

§ 371 Date: Jul. 19, 2024

(30) Foreign Application Priority Data
Feb. 5, 2021 (JP) 2021-017497

(51) Int. Cl. A45D 20/12; A45D 44/00

(52) U.S. Cl. CPC A45D 20/12; A45D 2044/007

(57) ABSTRACT

A hair dryer as a hair care device includes heat application unit (30), component generation unit (40), part detection unit (64), and controller (80). Heat application unit (30) applies heat to a user's hair. Component generation unit (40) generates a component acting on hair. Part detection unit (64) detects at least a part of hair. Controller (80) estimates a part to which heat or the component is applied based on an output of part detection unit (64), and adjusts amount of heat set in heat application unit (30) or a component amount set in component generation unit (40) for each part.

### TECHNICAL FIELD

The present disclosure relates to a hair care device.

### BACKGROUND ART

Conventionally, there is a hair care device such as a hair dryer that not only merely dries a user's hair but also applies an effective component for the user's hair. For example, PTL 1 discloses a technique related to a hair dryer in which ions are applied to hair as an effective component, and an amount of the component is adjusted based on usage time in addition to settings by the user.

### CITATION LIST

### Patent Literature

PTL 1: Unexamined Japanese Patent Publication No. 2019-58484

### SUMMARY OF THE INVENTION

Hairstyles and hair quality are different user by user. Further, hair quality in the hair of individual users is different for each part. Therefore, even when amount of ions as an effective component for hair is adjusted as in the hair dryer disclosed in PTL 1, it may not act effectively depending on the part of the user's hair. In other words, the hair dryer disclosed in PTL 1 does not necessarily achieve a finish of hair desired by the user for each part.

The present disclosure provides a hair care device that easily provides a finish of hair desired by a user.

A hair care device according to one aspect of the present disclosure includes a heat application unit that applies heat to a user's hair, a component generation unit that generates a component acting on the hair, and a part detection unit that detects at least a part of the hair. Further, the hair care device according to one aspect of the present disclosure includes a controller that estimates a part to which heat or the component is applied based on an output of the part detection unit, and adjusts, for each part, amount of heat set in the heat application unit or a component amount set in the component generation unit.

According to the present disclosure, it is possible to provide a hair care device that easily provides a finish of hair desired by a user.

### BRIEF DESCRIPTION OF THE DRAWINGS

FIG. 1 is a schematic perspective view illustrating a configuration of a hair dryer according to a first exemplary embodiment.

FIG. 2 is a schematic sectional view illustrating the configuration of the hair dryer according to the first exemplary embodiment.

FIG. 3A is a diagram illustrating a configuration of a first electrostatic atomization device as an example of a component generation device that can be adopted as a component generation unit.

FIG. 3B is a diagram illustrating a configuration of a second electrostatic atomization device as an example of a component generation device that can be adopted as a component generation unit.

FIG. 3C is a diagram illustrating a configuration of a third electrostatic atomization device as an example of a component generation device that can be adopted as a component generation unit.

FIG. 4A is a view related to a first installation position example of a wetting detection sensor and an illumination unit.

FIG. 4B is a view related to a second installation position example of the wetting detection sensor and the illumination unit.

FIG. 4C is a view related to a third installation position example of the wetting detection sensor and the illumination unit.

FIG. 5 is a block diagram illustrating a configuration of a controller of the hair dryer according to the first exemplary embodiment.

FIG. 6A is a flowchart illustrating an initialization operation for a part determination step.

FIG. 6B is a schematic view illustrating the initialization operation required by a user.

FIG. 7A is a view illustrating a state where the user turns on a power supply of the hair dryer.

FIG. 7B is a view illustrating a state where the user has started a drying operation.

FIG. 8A is a schematic view illustrating hair fluttering in a case where air is blown against the tip of hair.

FIG. 8B is a schematic view illustrating a case where air is not blown against hair or the like.

FIG. 9A is a view illustrating an angle from a horizontal line as an orientation of the hair dryer during the drying operation.

FIG. 9B is a view illustrating a left and right deflection angle as the orientation of the hair dryer during the drying operation.

FIG. 10 is a graph illustrating a variation example of information derived from an output of a part detection unit.

FIG. 11 is a timing chart illustrating an example of a relationship between an application amount of a cosmetic and hair detection.

FIG. 12 is a timing chart illustrating an example of a relationship between an application amount of charged fine particles and part detection.

FIG. 13 is a timing chart illustrating an example of a relationship between the application amount of the cosmetic and part detection.

FIG. 14 is a timing chart illustrating an example of a relationship between application amounts of two kinds of cosmetics and part detection.

FIG. 15 is a timing chart illustrating an example of a relationship between the application amount of the charged fine particles and detection of a permed portion.

FIG. 16 is a timing chart illustrating an example of a relationship between an air volume and part detection.

FIG. 17A is a view illustrating a first input screen of a first example of an input screen.

FIG. 17B is a view illustrating a second input screen of the first example of the input screen.

FIG. 17C is a view illustrating a third input screen of the first example of the input screen.

FIG. 18A is a view illustrating a first example of an output screen at the time when the middle of hair is dried.

FIG. 18B is a view illustrating a first example of the output screen at the time when the root of hair is dried.

FIG. 19 is a view illustrating a second example of the output screen.

FIG. 20A is a view illustrating a third example of the output screen.

FIG. 20B is a view illustrating the third example of the output screen.

FIG. 21 is a view illustrating a fourth example of the output screen.

FIG. 22A is a view illustrating a first input screen of a second example of the input screen.

FIG. 22B is a view illustrating a second input screen of the second example of the input screen.

FIG. 23 is a table illustrating a setting example in a case where the component amount is changed for each part of hair.

FIG. 24 is a table illustrating a principle for determining whether or not hair is wet.

FIG. 25 is a table illustrating criteria for determining whether or not hair is wet.

FIG. 26 is a schematic perspective view illustrating a configuration of a hair dryer according to a second exemplary embodiment.

### DESCRIPTION OF EMBODIMENT

Hereinafter, a hair care device according to an exemplary embodiment of the present disclosure will be described in detail with reference to the drawings. However, unnecessary detailed description may be omitted. For example, detailed descriptions of already well-known matters or redundant descriptions of substantially the same configuration may be omitted. Note that, the accompanying drawings and the following description are merely presented to help those skilled in the art fully understand the present disclosure, and are not intended to limit the subject matters described in the scope of claims.

### First Exemplary Embodiment

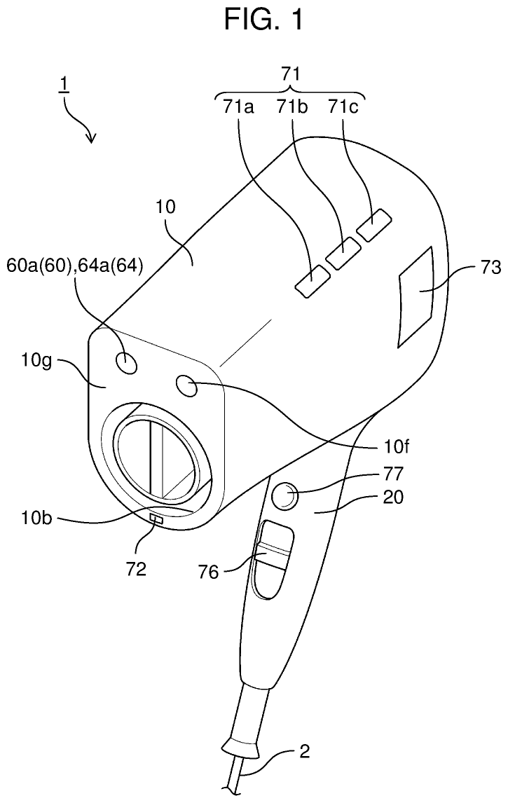

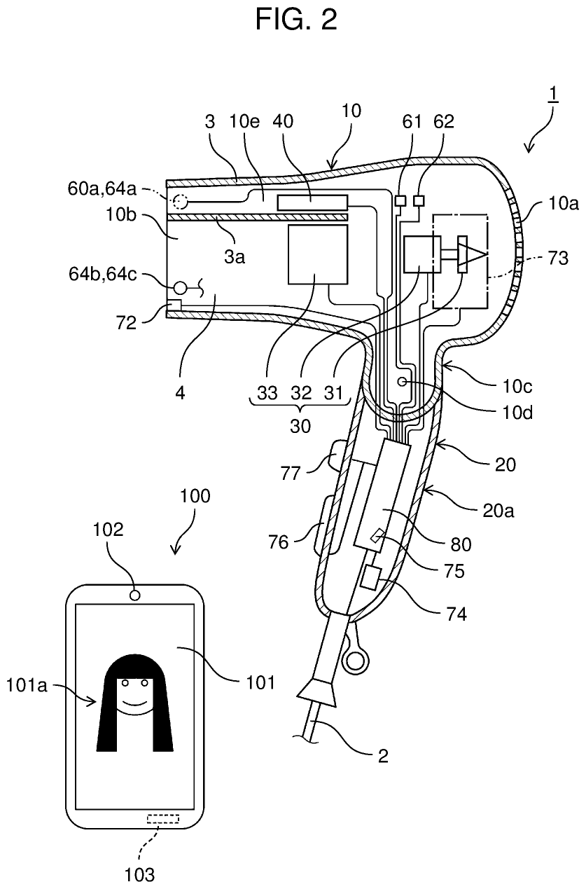

FIG. 1 is a schematic perspective view illustrating a configuration of hair dryer 1 as a hair care device according to the first exemplary embodiment. Hair dryer 1 includes main body 10 that sends warm air toward a user, and grip part 20 as a portion gripped by the user's hand during use. FIG. 2 is a schematic sectional view illustrating the configuration of hair dryer 1 cut along an air blowing direction so as to include main body 10 and grip part 20.

Main body 10 includes housing 3 forming an outer wall in which a plurality of divided bodies are joined together. Inside housing 3, air blowing flow channel 4 is formed from suction port 10a provided at one end portion in a longitudinal direction to discharge port 10b provided at the other end portion. As shown in FIG. 2, main body 10 and grip part 20 are pivotably connected by connecting part 10c with respect to connecting shaft 10d. For example, when hair dryer 1 is not in use, grip part 20 is folded against main body 10 so as to be substantially parallel to an axial direction of main body 10 extending in the air blowing direction. In grip part 20, power supply cord 2 is pulled out from an end portion opposite to connecting part 10c.

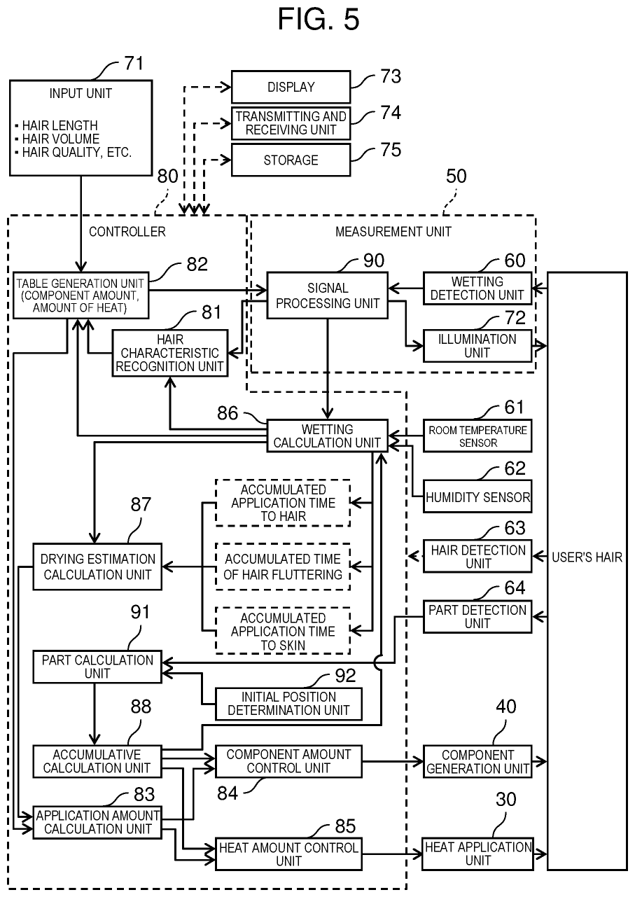

To start with, hair dryer 1 includes heat application unit 30, component generation unit 40, measurement unit 50 (see FIG. 5), input unit 71, and display 73.

Heat application unit 30 applies heat to the user's hair. In the present exemplary embodiment, heat application unit 30 is an air blower that generates warm air to be sent to the user's hair. Heat application unit 30 includes, for example, fan 31, motor 32, and heating unit 33. Fan 31 is disposed on an upstream side in air blowing flow channel 4 and rotates when motor 32 is driven. When fan 31 rotates, an air flow is formed to flow into air blowing flow channel 4 from outside via suction port 10a, pass through air blowing flow channel 4, and is discharged from discharge port 10b to outside. Heating unit 33 is disposed downstream of fan 31 and heats the air flow sent from fan 31. When heating unit 33 is operated, the air flow formed by fan 31 is heated, and warm air is blown out from discharge port 10b. Heating unit 33 may be, for example, a heater in which an electric resistor having a strip shape and a corrugated plate shape is wound along an inner periphery of housing 3.

Component generation unit 40 generates a component that acts on the user's hair. Here, the component that acts on hair refers to a so-called beauty component that can effectively act on at least hair quality of the user. Examples of the component include, for example, an agent or an organic substance, negative ions, metal fine particles, and charged fine particle water. The agent or organic substance is, for example, a moisturizing component (a moisturizing agent), a repairing component (a repairing agent), a coating component (a coating agent), or a treatment component (a treatment agent). The moisturizing component is, for example, 1,3-butylene glycol, glycerin, panthenol, ceramide, hyaluronic acid, honey, or a polysaccharide. The repair component is, for example, hydrolyzed collagen, hydrolyzed keratin, an amino acid, hair protection protein, a polypeptide, cholesterol, a cationic surfactant or an organic acid. The coating component is, for example, silicon, squalane or an oily component. The treatment component is, for example, a cationic surfactant, an amino acid, a polypeptide, panthenol, or ceramide. Further, the charged fine particle water is an electrically charged nano-sized water particle containing OH radicals.

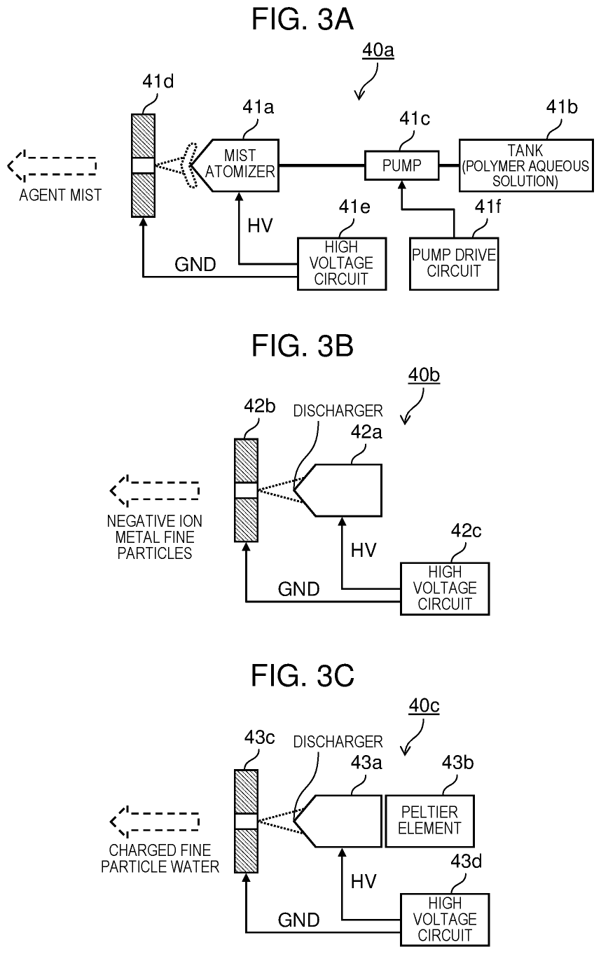

FIGS. 3A to 3C are schematic diagrams illustrating configurations of various component generation devices that can be adopted as component generation unit 40. FIG. 3A is a diagram illustrating a configuration of first electrostatic atomization device 40a as an example of an agent spraying device that uses an agent or an organic substance as a component acting on hair. First electrostatic atomization device 40a includes mist atomizer 41a, tank 41b, pump 41c, GND electrode 41d, high-voltage circuit 41e, and pump drive circuit 41f. Mist atomizer 41a is a discharger formed to hold a liquid as an agent or an organic substance. Tank 41b stores an aqueous solution containing, for example, a polymer as an agent or an organic substance. Pump 41c is installed in a pipe connecting tank 41b and mist atomizer 41a, and sends a polymer aqueous solution stored in tank 41b to mist atomizer 41a. High-voltage circuit 41e applies a high voltage (HV) to mist atomizer 41a. Pump drive circuit 41f controls driving of pump 41c. High-voltage circuit 41e and pump drive circuit 41f are controlled by component amount control unit 84 (see FIG. 5) in controller 80 to be described in detail below. When a high voltage is applied between mist atomizer 41a and GND electrode 41d, corona discharge or the like occurs, and an agent mist containing a polymer is generated by this discharge action. Note that, the agent spraying device that uses an agent or an organic substance as the component acting on hair is not limited to an electrostatic atomization device such as first electrostatic atomization device 40a, and may be an ultrasonic atomization device, a centrifugal pump, or the like.

FIG. 3B is a diagram illustrating a configuration of second electrostatic atomization device 40b as an example of a component generation device that use negative ions or metal fine particles as a component acting on hair. Second electrostatic atomization device 40b includes discharger 42a. GND electrode 42b, and high-voltage circuit 42c. High-voltage circuit 42c is controlled by component amount control unit 84 in the same manner as the configuration of first electrostatic atomization device 40a. When a high voltage is applied between discharger 42a and GND electrode 41d, for example, corona discharge or the like occurs, and negative ions negatively charged based on moisture in the air are generated by this discharge action.

FIG. 3C is a diagram illustrating a configuration of third electrostatic atomization device 40c as an example of a component generation device that uses charged fine particle water as a component acting on hair. Third electrostatic atomization device 40c includes discharger 43a, Peltier element 43b as a dew condensation unit, GND electrode 43c, and high-voltage circuit 43d. High-voltage circuit 43d is controlled by component amount control unit 84 in the same manner as the configuration of first electrostatic atomization device 40a. When a high voltage is applied between discharger 43a and GND electrode 43c, corona discharge or the like occurs, and charged fine particle water based on moisture in the air are generated by this discharge action.

For example, when component generation unit 40 in the present exemplary embodiment is third electrostatic atomization device 40c, as shown in FIG. 2, partition plate 3a that forms branch channel 10e extending in parallel with air blowing flow channel 4 is installed inside housing 3 of main body 10. Air blowing flow channel 4 allows an air flow passing through heating unit 33 to flow, whereas branch channel 10e allows an air flow not passing through heating unit 33 to flow. Then, third electrostatic atomization device 40c is installed in branch channel 10e. Further, front surface portion 10g that is a part of main body 10 and faces hair H during, for example, a drying operation, has component discharge port 10f. Component discharge port 10f communicates with branch channel 10e, and discharges the component generated by component generation unit 40 to outside. Note that, component generation unit 40 may be at least one of first electrostatic atomization device 40a, second electrostatic atomization device 40b, and third electrostatic atomization device 40c. In other words, a plurality of component generation units 40 may be provided for each component to be applied.

Measurement unit 50 (see FIG. 5) measures or photographs the user's hair, and transmits signal-processed information to controller 80. In the present exemplary embodiment, measurement unit 50 adopts a configuration that measures the user's hair. In this case, measurement unit 50 includes wetting detection unit 60, illumination unit 72, and signal processing unit 90 (see FIG. 5).

Wetting detection unit 60 detects the parameters that can be referred to for obtaining information on wetting of the user's hair. In the present exemplary embodiment, wetting detection unit 60 is wetting detection sensor 60a having at least an absorption wavelength (for example, 1450 nm) of water as a hair measurement value. Specifically, wetting detection sensor 60a may be a photodiode. Illumination unit 72 is a component paired with wetting detection sensor 60a that is, for example, a photodiode, and emits light having at least an absorption wavelength of water. Note that, signal processing unit 90 will be described in conjunction with the following matters related to controller 80.

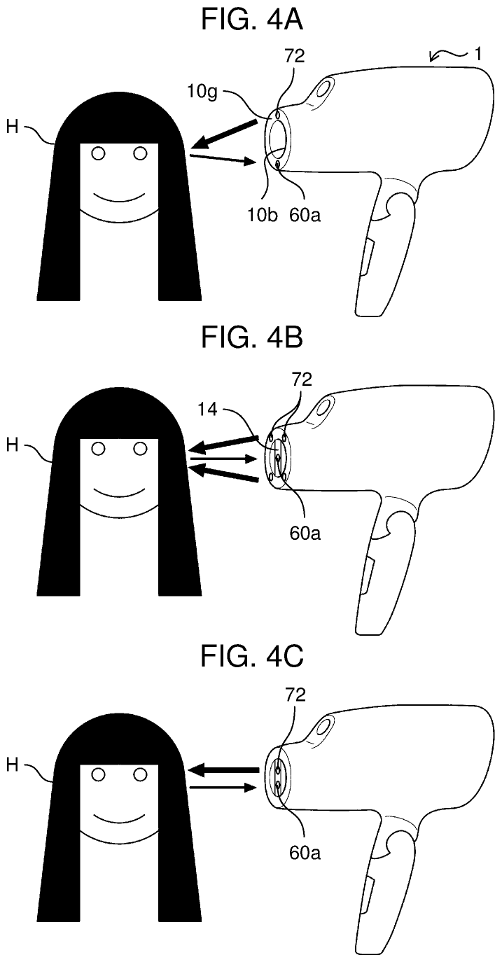

FIGS. 4A to 4C are schematic diagrams for explaining a relationship between each installation position of wetting detection sensor 60a and illumination unit 72. Each installation position of wetting detection sensor 60a and illumination unit 72 is an example in the case shown in FIGS. 1 and 2, and specifically, a plurality of examples as shown in FIGS. 4A to 4C are conceivable. While illumination unit 72 is a light irradiation unit, wetting detection sensor 60a is a light receiving unit that receives light emitted from illumination unit 72 and then reflected by the hair H of the user. Wetting detection sensor 60a and illumination unit 72 are installed on front surface portion 10g or nozzle part 14 (see FIGS. 4B and 4C) attached to discharge port 10b.

FIG. 4A is a diagram related to a first installation position example of wetting detection sensor 60a and illumination unit 72. In the first installation example, there is one wetting detection sensor 60a and one illumination unit 72. Wetting detection sensor 60a is installed on a part of front surface portion 10g. Illumination unit 72 is installed on a part of front surface portion 10g opposite to wetting detection sensor 60a across discharge port 10b. In this case, since wetting detection sensor 60a and illumination unit 72 are separated from each other in a distance larger than or equal to an opening diameter of discharge port 10b, an incident angle and a reflection angle of light are large.

FIG. 4B is a diagram related to a second installation position example of wetting detection sensor 60a and illumination unit 72. In the second installation example, there is one wetting detection sensor 60a, but there are a plurality of illumination units 72. Wetting detection sensor 60a is installed on nozzle part 14 so as to be positioned approximately at a center of discharge port 10b. There are, for example, four illumination units 72, installed at equal intervals from each other on front surface portion 10g. In this case, since wetting detection sensor 60a and illumination unit 72 are separated from each other to a certain extent, it is possible to increase an irradiation amount of light while ensuring the incident angle and the reflection angle of light at a certain size.

FIG. 4C is a diagram related to a third installation position example of wetting detection sensor 60a and illumination unit 72. In the third installation example, wetting detection sensor 60a and illumination unit 72 are installed one by one on nozzle part 14. In this case, since wetting detection sensor 60a and illumination unit 72 are relatively close to each other, the incident angle and the reflection angle of light are small.

Hereinafter, in the present exemplary embodiment, as an example, a description will be given on an assumption that wetting detection sensor 60a and illumination unit 72 are installed based on the second installation example shown in FIG. 4B.

Input unit 71 is, for example, a button for the user to input information on characteristics of the user's hair (hereinafter, referred to as “hair characteristics”). Here, the hair characteristics refer to at least one of a hairstyle of a user, a length of hair, a volume of hair (hair amount), and hair quality related to the thickness or gloss of hair. In the example shown in FIG. 1, input unit 71 includes three input buttons, that is, hair quality input unit 71a, hair length input unit 71b, and hair volume input unit 71c, installed in housing 3, respectively. Note that, input unit 71 may also include a button for simply switching an air volume, an air temperature, and the like according to the user's preference.

Display 73 is, for example, a touch panel type display screen installed in housing 3, and functions as an input screen on which a user inputs information or an output screen that displays information to the user. Note that, a state of functioning as an input screen or an output screen will be described in detail below. Further, in a case where display 73 functions as an input screen, display 73 substitutes the function performed by input unit 71, so that input unit 71 may be unnecessary in some cases.

Further, as shown in FIG. 2, hair dryer 1 includes room temperature sensor 61, humidity sensor 62, hair detection unit 63, and part detection unit 64.

Room temperature sensor 61 is a sensor for measuring a temperature in a room where hair dryer 1 is used. Room temperature sensor 61 is installed inside housing 3. An output signal from room temperature sensor 61 is transmitted to controller 80.

Humidity sensor 62 is a sensor for measuring humidity in a room where hair dryer 1 is used. Room temperature sensor 61 is installed inside housing 3. An output signal from humidity sensor 62 is transmitted to controller 80.

Hair detection unit 63 detects whether the user has hair. Hair detection unit 63 is, for example, a laser range finder or a time of flight (ToF) camera, and is installed in a part of front surface portion 10g. An output signal from hair detection unit 63 is transmitted to controller 80.

Part detection unit 64 detects a part to which heat is applied by heat application unit 30 or a part to which a component is applied by component generation unit 40. Part detection unit 64 is at least one of distance measurement unit 64a, orientation detection unit 64b, and surface condition detection unit 64c. An output signal from part detection unit 64 is transmitted to controller 80.

Distance measurement unit 64a is a distance sensor that measures a distance to the hair or the user's skin (face). In a case where part detection unit 64 is distance measurement unit 64a, distance measurement unit 64a is installed on a part of front surface portion 10g. Here, in the present exemplary embodiment, wetting detection sensor 60a, which is, for example, a photodiode, is provided as wetting detection unit 60. Therefore, in such a case, distance measurement unit 64a as part detection unit 64 may be shared with wetting detection sensor 60a as shown in FIG. 1. Hereinafter, a description will be given of the present exemplary embodiment, on the assumption that a certain photodiode functions as wetting detection sensor 60a and distance measurement unit 64a. Note that, distance measurement unit 64a may be provided independently separately from wetting detection sensor 60a.

Orientation detection unit 64b is an orientation sensor with at least one axis that detects a position or an orientation of hair dryer 1. Note that, in a case where part detection unit 64 is orientation detection unit 64b, orientation detection unit 64b is not limited to be installed on front surface portion 10g, and may be installed inside housing 3.

Surface condition detection unit 64c is, for example, a laser range finder or a time of flight (ToF) camera that detects a surface condition of the hair. Regarding surface condition detection unit 64c, in a case where part detection unit 64 is surface condition detection unit 64c, surface condition detection unit 64c is installed on a part of front surface portion 10g.

FIG. 5 is a block diagram showing a configuration of controller 80 of hair dryer 1. Controller 80 controls overall operations of hair dryer 1, and at least controls operations of heat application unit 30 and component generation unit 40 based on the hair measurement value obtained from measurement unit 50. Controller 80 is installed, for example, inside housing 20a of grip part 20. Note that, controller 80 includes a computer system including a processor and a memory. Then, when the processor executes programs stored in the memory, the computer system functions as controller 80. Here, the programs executed by the processor is recorded in advance in the memory of the computer system, but may be provided by being recorded in a non-transitory recording medium such as a memory card, or may be provided through a telecommunication line such as the Internet.

To start with, controller 80 includes hair characteristic recognition unit 81, table generation unit 82, application amount calculation unit 83, component amount control unit 84, and heat amount control unit 85. Hair characteristic recognition unit 81, table generation unit 82, application amount calculation unit 83, component amount control unit 84, and heat amount control unit 85 are a block group for determining a component application amount and a heat application amount based on the hair characteristics of the user.

Hair characteristic recognition unit 81 classifies the hair characteristics of the user based on the hair measurement value obtained from measurement unit 50.

Table generation unit 82 sets the component amount of the component generated by component generation unit 40 and the amount of heat applied from heat application unit 30, and manages these set values as a table. In table generation unit 82, the component amount and the amount of heat are set for each hair characteristic classified by hair characteristic recognition unit 81.

Based on the component amount or the amount of heat set by table generation unit 82, application amount calculation unit 83 calculates the component application amount applied to hair by component generation unit 40 or the heat application amount applied to hair by heat application unit 30. In the present exemplary embodiment, application amount calculation unit 83 can execute the following two types of calculations. First, application amount calculation unit 83 calculates the component application amount for each user based on the hair characteristics of the whole hair classified by hair characteristic recognition unit 81 and the component amount set by table generation unit 82. Second, application amount calculation unit 83 calculates the component application amount or the heat application amount for each part of the hair based on the hair characteristics for each part of the hair classified by hair characteristic recognition unit 81 and the component amount set by table generation unit 82.

Component amount control unit 84 controls the operation of component generation unit 40, that is, the component amount of the component generated by component generation unit 40, based on the component application amount transmitted from application amount calculation unit 83.

Heat amount control unit 85 controls the operation of heat application unit 30, that is, the amount of heat applied from heat application unit 30 based on the heat application amount transmitted from application amount calculation unit 83.

Further, controller 80 includes wetting calculation unit 86 and drying estimation calculation unit 87. Wetting calculation unit 86 and drying estimation calculation unit 87 are a block group for reflecting a dry state of the user's hair in the component application amount and the heat application amount.

Wetting calculation unit 86 calculates wetting information about wetting of the user's hair based on the hair measurement value obtained from measurement unit 50. Here, for example, in a case where wetting detection unit 60 in measurement unit 50 is wetting detection sensor 60a, the wetting information is absorbance calculated based on signal intensity from wetting detection sensor 60a.

Drying estimation calculation unit 87 estimates the degree of dryness of the user's hair based on the wetting information calculated by wetting calculation unit 86. When the wetting information is the absorbance, drying estimation calculation unit 87 estimates the degree of dryness based on a change in absorbance. In drying estimation calculation unit 87, for example, an accumulated time (time subtraction) in which a component or heat is applied to the hair, an accumulated time (time addition) in which the hair is in a fluttering state, an accumulated time (time addition) in which a component or heat is applied to the skin (face), or the like is appropriately referred to according to the change in absorbance. The degree of dryness estimated by drying estimation calculation unit 87 is reflected in the component application amount by component amount control unit 84 or the heat application amount by heat amount control unit 85 via application amount calculation unit 83. In other words, the component application amount or the heat application amount is corrected for each degree of dryness reflecting various accumulated times.

Further, controller 80 includes part calculation unit 91, initial position determination unit 92, and accumulative calculation unit 88. Part calculation unit 91, initial position determination unit 92, and accumulative calculation unit 88 are a block group for specifying a part of the user's hair to which the component and heat are applied.

Part calculation unit 91 estimates a part of the hair or the skin to which heat from heat application unit 30 is applied or to which the component from component generation unit 40 is applied, based on the output from part detection unit 64 and an initial position determined by initial position determination unit 92.

Initial position determination unit 92 determines an initial position of hair dryer 1, and transmits the initial position to part calculation unit 91. Note that, an initial position determination step performed by initial position determination unit 92 will be described in detail below.

Accumulative calculation unit 88 calculates, for each part estimated by part calculation unit 91, an accumulative heat amount that is an accumulative amount of heat applied by heat application unit 30 or an accumulative component amount that is an accumulative amount of the component applied by component generation unit 40. In this case, heat amount control unit 85 causes heat application unit 30 to adjust the amount of heat based on the accumulative amount of heat calculated by accumulative calculation unit 88. Specifically, heat amount control unit 85 corrects the heat application amount using data related to the accumulative amount of heat calculated by accumulative calculation unit 88, and controls the operation of heat application unit 30. On the other hand, component amount control unit 84 causes component generation unit 40 to adjust the component amount based on the accumulative component amount calculated by accumulative calculation unit 88. Specifically, component amount control unit 84 corrects the component application amount using data related to the accumulative component amount calculated by accumulative calculation unit 88, and controls the operation of component generation unit 40.

Furthermore, controller 80 is electrically connected to signal processing unit 90 included in measurement unit 50. Signal processing unit 90 controls light irradiation by illumination unit 72, processes an output of wetting detection unit 60 that is wetting detection sensor 60a, and transmits the processed output to wetting calculation unit 86 as signal intensity. Further, signal processing unit 90 may transmit the output of wetting detection unit 60 to hair characteristic recognition unit 81 as signal intensity. In this case, hair characteristic recognition unit 81 can classify the hair characteristics of the user based on the signal intensity transmitted from signal processing unit 90.

Further, as shown in FIG. 2, hair dryer 1 includes power supply switch 76 and initialization switch 77. Power supply switch 76 and initialization switch 77 are installed, for example, in housing 20a of grip part 20. When a user operates power supply switch 76 to turn a power supply ON, power is supplied to each part of hair dryer 1 via power supply cord 2 extending from an end portion of grip 20. Further, power supply switch 76 can also operate switching between warm air and cold air by heat application unit 30, switching of the air volume, and the like. Initialization switch 77 is a switch used during initialization of a part determination step in which controller 80 determines a part of the user's hair during the drying operation.

Furthermore, hair dryer 1 may include transmitting and receiving unit 74 and storage 75.

Transmitting and receiving unit 74 transmits signals to a communication device outside hair dryer 1 or receives signals transmitted from the communication device outside hair dryer 1 in accordance with a command from controller 80. Here, the communication device outside may be, for example, portable terminal device 100 as shown in FIG. 2. Portable terminal device 100 includes terminal display unit 101, terminal photographing unit 102, and terminal communication unit 103. Terminal display unit 101 is a touch panel screen that displays image 101a. Terminal display unit 101 is an output screen that displays information to the user, and is an input screen on which the user instructs or inputs information by touching. Terminal communication unit 103 performs transmission and reception with at least transmitting and receiving unit 74 of hair dryer 1.

Storage 75 is an information storage medium that passes various data with controller 80 and stores these data. The type of the information storage medium is not particularly limited.

Next, an operation of hair dryer 1 will be described.

As a basic operation of hair dryer 1, when the user turns the power supply ON by operating power supply switch 76 while gripping grip part 20, heat application unit 30 starts to operate. Specifically, when motor 32 is driven by electric power supply and fan 31 rotates, air is taken into air blowing flow channel 4 from suction port 10a. At the same time, when heating unit 33 generates heat, the air sent from fan 31 is heated. The heated air becomes warm air and is discharged from discharge port 10b. Further, when the user appropriately operates input unit 71, hair dryer 1 causes component generation unit 40 to generate an effective component for hair and discharge it from component discharge port 10f.

Furthermore, hair dryer 1 automatically optimizes the component application amount for each part of the users hair. Hereinafter, optimization of the component application amount will be specifically described.

To start with, a description will be given of the part determination step in which controller 80 determines a part of the user's hair during the drying operation.

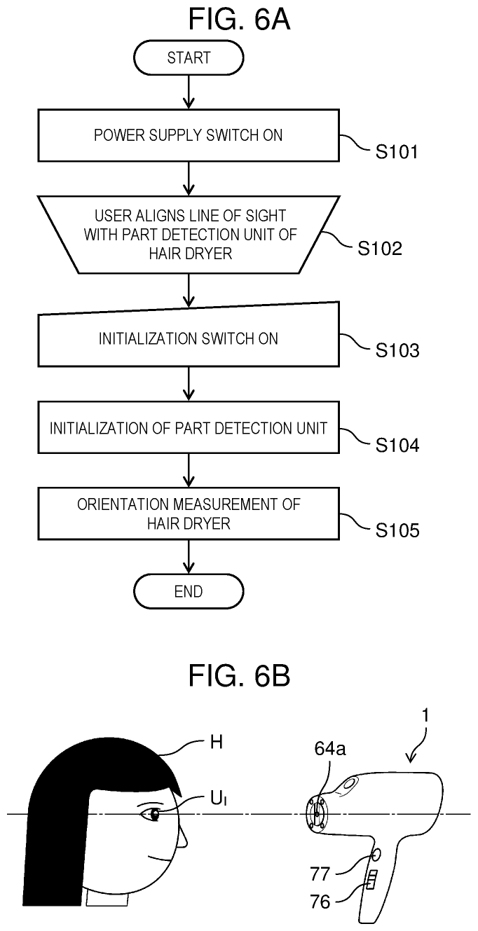

FIGS. 6A and 6B are drawings for illustrating an initialization operation performed before the part determination step is executed. In this example, part detection unit 64 used in the part determination step is at least distance measurement unit 64a shared with wetting detection sensor 60a shown in FIG. 4B.

FIG. 6A is a flowchart illustrating an initialization operation for the part determination step. In a case where the user desires to perform a component applying operation (the drying operation) that automatically optimizes the component application amount for each part of the hair, the user first turns on power supply switch 76 and turns on hair dryer 1 (step S101). Next, the user aligns his or her line of sight with distance measurement unit 64a (step S102).

FIG. 6B is a schematic view illustrating an initial operation required by the user in step S102. The user positions the front of hair dryer 1 in front of his or her face, and aligns line of sight Uₗ and distance measurement unit 64a on a horizontal plane.

Next, the user turns on initialization switch 77 while line of sight Uₗ and distance measurement unit 64a are aligned with each other (step S103).

Next, controller 80 initializes part detection unit 64 (step S104). Since part detection unit 64 is at least distance measurement unit 64a in this description herein, controller 80 determines the distance from distance measurement unit 64a to line of sight Uₗ as initialization of distance measurement unit 64a. On the other hand, for example, there may be a case where orientation detection unit 64b exists as part detection unit 64 other than distance measurement unit 64a. In this case, orientation detection unit 64b may be initialized by causing orientation detection unit 64b to measure the orientation of hair dryer 1 subsequent to or simultaneously with step S104 (step S105). In the initialization of orientation detection unit 64b, an initial orientation of orientation detection unit 64b is determined. The initialization operation ends at the end of step S104 or step S105.

Next, the principle of determining a part of the hair in the part determination step will be described.

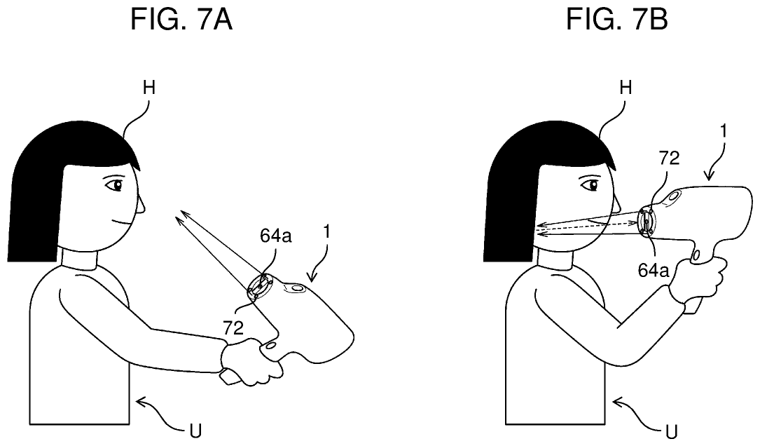

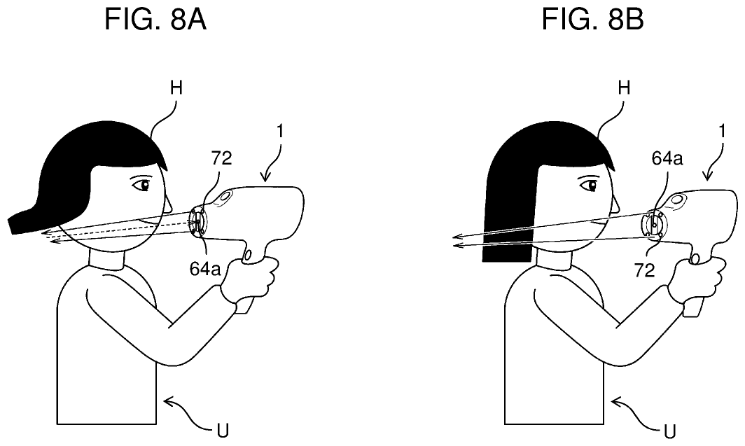

FIGS. 7A and 7B are schematic views illustrating a state from a time when the user turns on the power supply of hair dryer 1 to a time when the user starts the drying operation. FIG. 7A is a view illustrating a state where the user turns on the power supply of hair dryer 1. Arrow-headed solid lines in FIGS. 7A and 7B and FIGS. 8A and 8B below represent lights emitted from a plurality of illumination units 72. At this time, since the user operates power supply switch 76 while looking at hair dryer 1, the lights emitted from illumination units 72 are not reflected at any part.

FIG. 7B is a view illustrating a state where the user has started the drying operation for drying hair using hair dryer 1. When the user turns discharge port 10b (see FIGS. 1 and 2) of hair dryer 1 toward his or her hair, light emitted from illumination unit 72 is reflected by the hair or the skin (face), and distance measurement unit 64a detects the reflected light. Arrow-headed broken lines in FIG. 7B and FIG. 8A below represent the reflected light on the hair or the skin.

FIGS. 8A and 8B are schematic views illustrating hair fluttering during the drying operation. FIG. 8A is a schematic view illustrating hair fluttering in a case where air from hair dryer 1 is blown against the tip of the user's hair. When air is blown against the tip of the hair, the hair flutters more than the hair when air is blown against the middle of the hair. Similarly, when air is blown against the middle of the hair, the hair flutters more than the hair when air is blown against the root of the hair. In other words, when the tip, the middle, and the root of the hair are compared with one another, the hair flutters the least when air is blown against the root.

FIG. 8B is a schematic view illustrating a case where air from hair dryer 1 is not blown against the user's hair or the like because the direction toward which discharge port 10b is directed is shifted from the user's hair or skin. In this case, the user's hair does not flutter. Further, since the light emitted from illumination unit 72 is not reflected at any part, distance measurement unit 64a does not detect the reflected light.

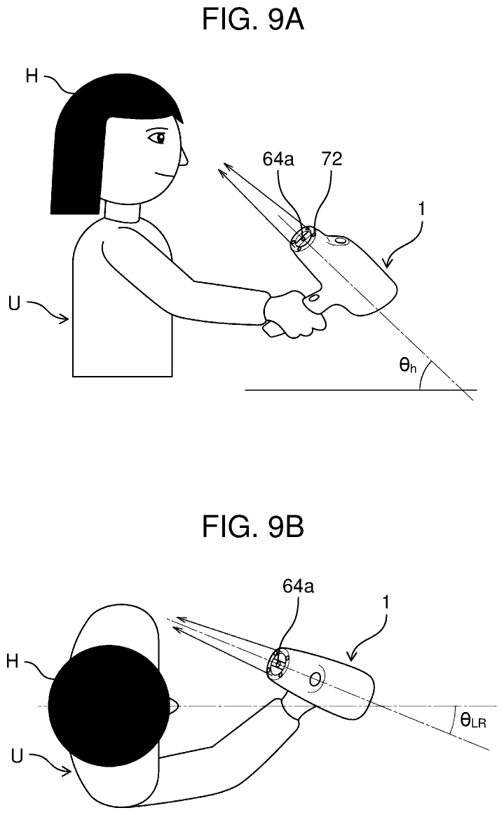

FIGS. 9A and 9B are schematic views illustrating an orientation of hair dryer 1 during the drying operation. FIG. 9A shows an orientation of hair dryer 1 inclined at angle θₕ from the horizontal line. In step S105 during the initialization operation shown in FIG. 6A, it is assumed that the initial orientation of hair dryer 1 in this case is set when angle θₕ from the horizontal line becomes approximately 0°.

FIG. 9B shows the orientation of hair dryer 1 in which distance measurement unit 64a is deflected leftward and rightward at angle θ_{LR} on the horizontal plane from the initial orientation toward the front of the line of sight of the user. In step S105 during the initialization operation shown in FIG. 6A, it is assumed that the initial orientation of hair dryer 1 in this case is set when a left and right deflection angle θ_{LR} becomes approximately 0°.

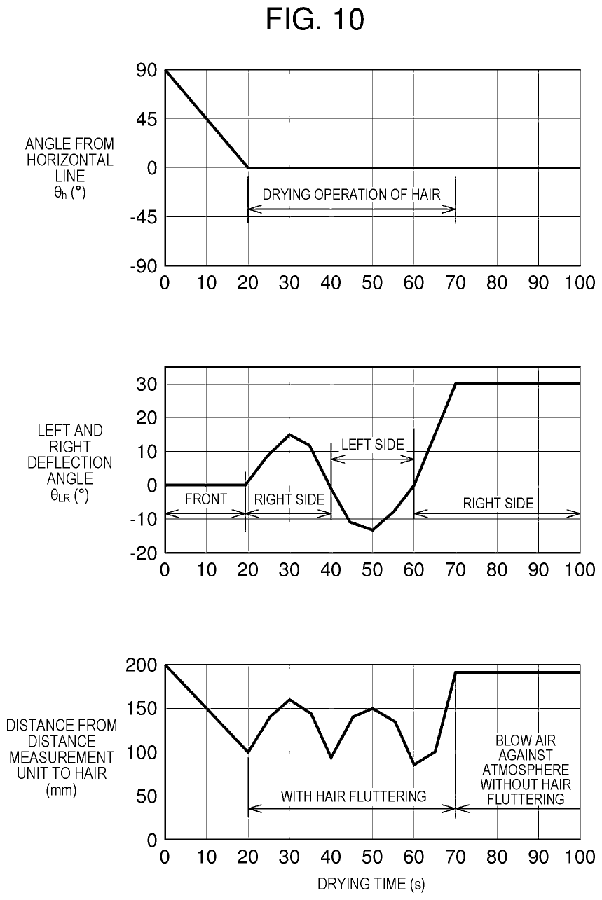

FIG. 10 is a graph illustrating a variation example of information derived from an output of part detection unit 64. An upper diagram shows, as an orientation of hair dryer 1, angle θₕ(°) from the horizontal line relative to a drying time (seconds). A middle diagram shows, as an orientation of hair dryer 1, a left and right deflection angle θ_{LR}(°) relative to the drying time (seconds). A lower diagram shows the distance (mm) from distance measurement unit 64a to the hair relative to the drying time (seconds). In FIG. 10, the drying times, which are a horizontal axe in the upper diagram, the middle diagram, and the lower diagram, correspond to one another. Further, in the following diagrams, the drying time means a drying operation time during which hair dryer 1 operates for drying.

First, a case is assumed that part detection unit 64 is orientation detection unit 64b. In this case, referring to the upper diagram of FIG. 10, the angle θₕ from the horizontal line is not 0° for a while after the drying operation of hair dryer 1 is started, and the orientation of hair dryer 1 is not stable. Note that, in the example of the upper diagram of FIG. 10, a while mentioned here is an elapse of time of 20 seconds since the start of the drying operation. When the orientation of hair dryer 1 is not stable, it can be estimated that discharge port 10b is not directed to the user's hair, so that it can be estimated that the user is not performing the drying operation.

Furthermore, in the example in the upper diagram of FIG. 10, after 20 seconds have elapsed since the start of the drying operation, the angle θₕ from the horizontal line becomes constant at 0°, and the orientation of hair dryer 1 is stable. When hair dryer 1 is stable in such an orientation, it can be estimated that discharge port 10b is directed to the user's hair. Further, with reference to the middle diagram or the lower diagram of FIG. 10 described below, it can be estimated that the user is performing the drying operation until 70 seconds further elapse after an elapse of time of 20 seconds from the start of the drying operation.

Therefore, in a case where part detection unit 64 is orientation detection unit 64b, part calculation unit 91 obtains a variation amount of angle θₕ from the horizontal with respect to the output of orientation detection unit 64b for a predetermined time after the start of the drying operation of hair dryer 1. Then, in a case where the variation amount is smaller than a predetermined reference amount, part calculation unit 91 determines that the user is performing the drying operation. On the other hand, in a case where the variation amount is larger than the predetermined reference amount, part calculation unit 91 determines that the user is not performing the drying operation. Here, the reference amount is an amount serving as a reference for determining whether the user is performing the drying operation, and may be set arbitrarily. For example, the reference amount of the angle θₕ herein may be in a range from −5° to +5°. In other words, when the variation amount of the angle θₕ is within the range from −5° to +5° while the user is operating hair dryer 1, part calculation unit 91 may determine that the drying operation is being performed since the variation amount of the angle θₕ is small. On the other hand, when the variation amount of the angle θₕ exceeds the range from −5° to +5° while the user is operating hair driver 1, part calculation unit 91 may determine that the drying operation is not being performed because the variation amount of the angle θₕ is large.

Second, a case is assumed that part detection unit 64 is orientation detection unit 64b as described above. In this case, referring to the middle diagram in FIG. 10, the left and right deflection angle θ_{LR} is approximately 0° for a while after the drying operation of hair dryer 1 is started. Note that, in the example in the middle diagram in FIG. 10, a while mentioned here is an elapse of time of 20 seconds since the start of the drying operation until 20. When the deflection angle θ_{LR} is 0°, it can be estimated that discharge port 10b is directed to the front of the user regardless of the value of the angle θₕ from the horizontal line.

Next, in the example in the middle diagram in FIG. 10, the deflection angle θ_{hR} is in a positive range from an elapse of time of 20 seconds since the start of the drying operation to an elapse of time of 40 seconds since the start of the drying operation. When the deflection angle θ_{LR} is in the positive range, it can be estimated that hair dryer 1 is deflected rightward and discharge port 10b is directed to the right side of the user's hair or skin (face). On the other hand, the deflection angle θ_{LR} is in a negative range from an elapse of time of 40 seconds since the start of the drying operation to an elapse of time of 60 seconds since the start of the drying operation. When the deflection angle θ_{LR} is in the negative range, it can be estimated that hair dryer 1 is deflected leftward, and discharge port 10b is directed to the left side of the user's hair or skin (face).

Furthermore, in the example in the middle diagram in FIG. 10, the deflection angle θ_{LR} changes to the positive range again after 60 seconds have elapsed since the start of the drying operation, and the deflection angle θ_{LR} becomes constant at 30° after 70 seconds have elapsed since the start of the drying operation. In this case, when hair dryer 1 is deflected rightward, discharge port 10b is directed to the right side of the user's hair or skin (face), and it can be estimated that the user is no longer performing the drying operation after 70 seconds have elapsed since the start of the drying operation.

Therefore, in a case where part detection unit 64 is orientation detection unit 64b, part calculation unit 91 obtains the variation amount of left and right deflection angle θ_{LR} with respect to the output of orientation detection unit 64b for a predetermined time after the start of the drying operation of hair dryer 1. Then, in a case where the variation amount of the deflection angle θ_{LR} is approximately 0° and constant, part calculation unit 91 may determine that discharge port 10b faces approximately frontward and the user is not performing the drying operation. Further, in a case where the deflection angle θ_{LR} is in the positive range and keeps varying, part calculation unit 91 may determine that discharge port 10b faces the right side of the user's hair or skin (face) and the user is performing the drying operation. On the other hand, in a case where the deflection angle θ_{LR} is in the negative range and keeps varying, part calculation unit 91 may determine that discharge port 10b faces leftward of the user's hair or skin (face) and the user is performing the drying operation. Furthermore, in a case where the deflection angle θ_{LR} is constant at an angle other than 0°, part calculation unit 91 may determine that the user is not performing the drying operation.

Thirdly, a case is assumed that part detection unit 64 is distance measurement unit 64a or surface condition detection unit 64c. Hereinafter, the description will be given of an example in which it is assumed that part detection unit 64 is distance measurement unit 64a, but the same applies to a case where part detection unit 64 is surface condition detection unit 64c. Further, in step S104 during the initialization operation shown in FIG. 6A, it is assumed that the distance from distance measurement unit 64a to line of sight UI at the initial position of distance measurement unit 64a is determined. In this case, referring to the lower diagram in FIG. 10, first, since the user is in a state where hair dryer 1 is brought close to the hair side for a while after the drying operation of hair dryer 1 is started, the distance from distance measurement unit 64a to the hair is shortened at an approximately constant speed. Note that, in the example in the lower diagram of FIG. 10, a while mentioned here is an elapse of time of 20 seconds since the start of the drying operation. While hair dryer 1 is brought close to the hair, discharge port 10b is not always directed to the user's hair, so that it can be estimated that the user is not performing the drying operation.

Next, in the example in the lower diagram in FIG. 10, until an elapse of time of 70 seconds since the start of the drying operation after an elapse of time of 20 seconds since the start of the drying operation, the distance from distance measurement unit 64a to the hair varies slightly. This is because, as shown in FIG. 8A, air from hair dryer 1 is blown against the hair to cause the hair to flutter, so that the position of the hair is not stable. In other words, when the position of the hair is not stable as described above, it can be estimated that the user is performing the drying operation.

Furthermore, in the example in the lower diagram in FIG. 10, after 70 seconds have elapsed since the start of the drying operation, the distance from distance measurement unit 64a to the hair does not vary in a relatively long state. This is because the user temporarily stops blowing air from hair dryer 1 against the hair, so that the hair does not flutter. Note that a state where air from hair dryer 1 is not blown against the hair is, in other words, a state where the user intends to direct air from hair dryer 1 to the atmosphere. In this time, in view of the relationship between the upper diagram and the middle diagram in FIG. 10, the drying operation of hair dryer 1 is continued as shown in FIG. 8B, but air blown from hair dryer 1 does not actually contribute to the drying of the hair, so that it can be estimated that the user is not performing the drying operation.

Therefore, in a case where part detection unit 64 is distance measurement unit 64a, part calculation unit 91 obtains the variation amount of the output of distance measurement unit 64a for a predetermined time after the start of the drying operation of hair dryer 1. Then, based on the magnitude of the variation amount, part calculation unit 91 determines a part against which air is blown from hair dryer 1. For example, in a case where the variation amount within a predetermined time is large, it can be considered that the hair is greatly fluttering. Therefore, it can be estimated that the part against which air is blown from hair dryer 1 is the tip of the user's hair. Similarly, in a case where the variation amount within the predetermined time is medium, it can be estimated that the part against which air is blown from hair dryer 1 is the middle of the user's hair. Further, in a case where the variation amount within the predetermined time is small, it can be considered that the hair is fluttering to a small extent. Therefore, it can be estimated that the part against which air is blown from hair dryer 1 is the root of the user's hair.

Hitherto, the part determination step has been described in which controller 80 determines the part of the user's hair during the drying operation, but the part against which air is blown from hair dryer 1 is synonymous with the part to which the component is applied from hair dryer 1. In other words, controller 80 automatically optimizes the component application amount for each part of the user's hair together with an example of the component application amount as described below.

Next, an example of timing related to the component applied by component generation unit 40 or the heat applied by heat application unit 30 with the drying time as a time series, will be described.

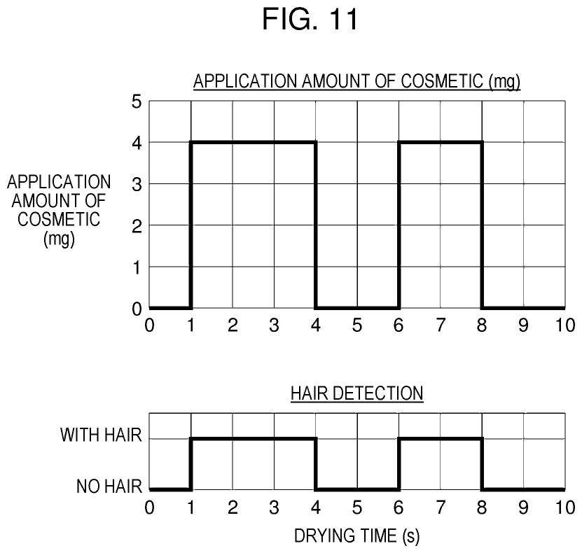

FIG. 11 is a timing chart illustrating a relationship between an application amount of a cosmetic and detection of the user's hair. An upper diagram shows the application amount (mg) of the cosmetic relative to the drying time (seconds). Hereinafter, the cosmetic is collectively referred to as various components illustrated above as an agent and an organic substance. Here, as an example, the application amount when the cosmetic is applied is constant at 4 mg. A lower diagram shows whether or not hair is detected relative to the drying time (seconds). In FIG. 11, the drying times, which are the horizontal axe in the upper diagram and the lower diagram, correspond to each other. Controller 80 determines existence or non-existence of hair based on, for example, the output signal of hair detection unit 63. Here, when hair is present, it means a case where air blown from hair dryer 1 blows against the users hair. On the other hand, when there is no hair, it means a case where air blown from hair dryer 1 does not blow against the user's hair. In other words, as shown in FIG. 11, controller 80 may cause component generation unit 40 to apply the cosmetic only when determining that hair is present.

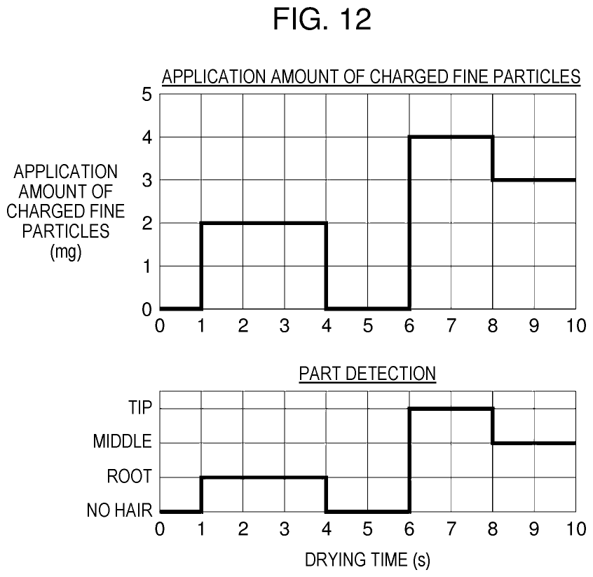

FIG. 12 is a timing chart illustrating an example of a relationship between the application amount of charged fine particles and part detection of the user's hair. The upper diagram shows the application amount (mg) of the charged fine particles relative to the drying time (seconds). The lower diagram shows part detection of the hair relative to the drying time (seconds). In FIG. 12, the drying times, which are the horizontal axe in the upper diagram and the lower diagram, correspond to each other. Controller 80 determines a part in contact with charged fine particle water discharged from hair dryer 1 based on, for example, the output signal of part detection unit 64. Here, as shown in FIG. 12, controller 80 does not cause component generation unit 40 to generate charged fine particle water while determining that there is no hair. On the other hand, controller 80 causes component generation unit 40 to apply charged fine particle water containing, for example, 2 mg of charged fine particles, while air is blown against the root of the hair to dry the hair. Further, controller 80 causes component generation unit 40 to apply charged fine particle water containing, for example, 3 mg of charged fine particles, while air is blown against the middle of the hair to dry the hair. Furthermore, controller 80 causes component generation unit 40 to apply charged fine particle water containing, for example, 4 mg of charged fine particles, while air is blown against the tip of the hair to dry the hair. In other words, controller 80 may decrease the application amount of the charged fine particle water to the root side of the hair and increase the application amount of the charged fine particle water to the tip side of the hair.

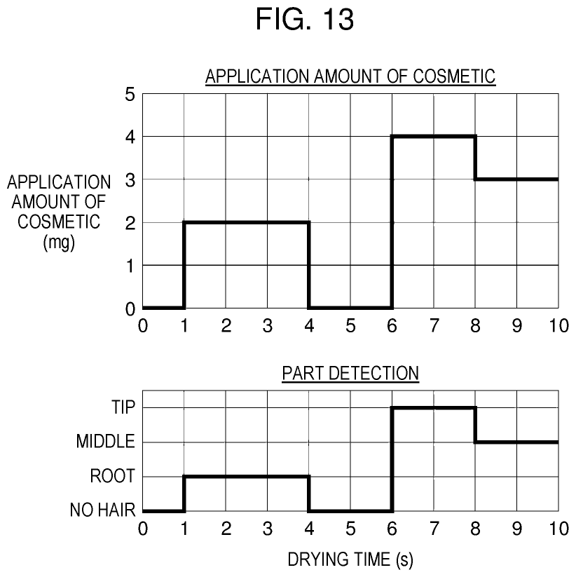

FIG. 13 is a timing chart illustrating an example of a relationship between the application amount of the cosmetic and part detection of the use's hair. An upper diagram shows the application amount (mg) of the cosmetic relative to the drying time (seconds). The lower diagram shows part detection of the hair relative to the drying time (seconds). In FIG. 13, the drying times, which are the horizontal axe in the upper diagram and the lower diagram, correspond to each other. Controller 80 determines a part in contact with the cosmetic discharged from hair dryer 1 based on, for example, the output signal of part detection unit 64. Here, as shown in FIG. 13, controller 80 does not cause component generation unit 40 to generate the cosmetic while determining that there is no hair. On the other hand, controller 80 causes component generation unit 40 to apply, for example, 2 mg of the cosmetic, while air is blown against the root of the hair to dry the hair. Further, controller 80 causes component generation unit 40 to apply, for example, 3 mg of the cosmetic, while air is blown against the middle of the hair to dry the hair. Furthermore, controller 80 causes component generation unit 40 to apply, for example, 4 mg of the cosmetic, while air is blown against the tip of the hair to dry the hair. In other words, controller 80 may decrease the application amount of the cosmetic to the root side of the hair and increase the application amount of the cosmetic to the tip side of the hair.

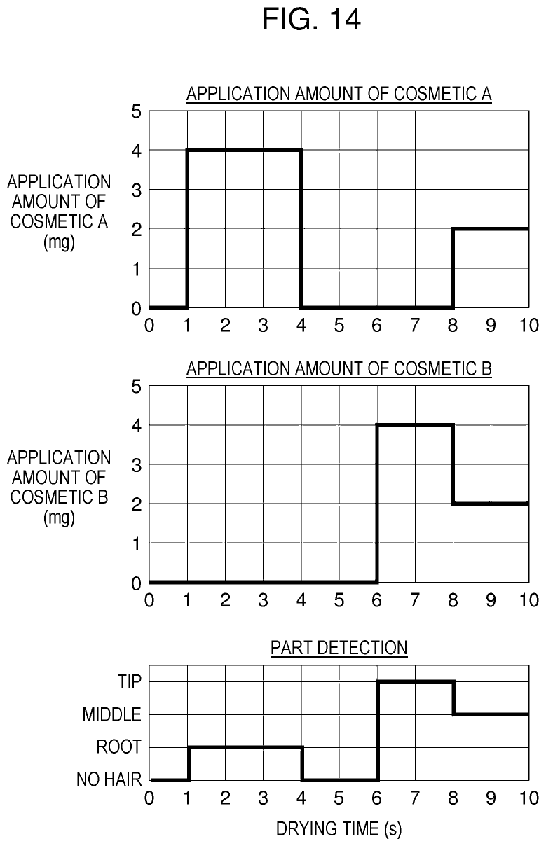

FIG. 14 is a timing chart illustrating an example of a relationship between application amounts of two kinds of cosmetics A and B and part detection of the user's hair. The upper diagram shows the application amount (mg) of the cosmetic A relative to the drying time (seconds). The middle diagram shows the application amount (mg) of the cosmetic B relative to the drying time (seconds). The cosmetic A and the cosmetic B are components different from each other. The cosmetic A is a component that effectively acts on hair characteristics, especially at the root of hair. The cosmetic B is a component that effectively acts on hair characteristics, especially at the tip of hair. The lower diagram shows part detection of the hair relative to the drying time (seconds). In FIG. 14, the drying times, which are the horizontal axe in the upper diagram, the middle diagram, and the lower diagram, correspond to one another. Controller 80 determines a part in contact with the cosmetic A or cosmetic B discharged from hair dryer 1 based on, for example, the output signal of part detection unit 64. Here, as shown in FIG. 14, controller 80 does not cause component generation unit 40 to generate either cosmetic A or cosmetic B while determining that there is no hair. On the other hand, controller 80 causes component generation unit 40 to apply, for example, only 4 mg of the cosmetic A, while air is blown against the root of the hair to dry the hair. Further, controller 80 causes component generation unit 40 to apply, for example, 2 mg of the cosmetic A and 2 mg of the cosmetic B, while air is blown against the middle of the hair to dry the hair. Furthermore, controller 80 causes component generation unit 40 to apply, for example, 4 mg of the cosmetic B, while air is blown against the tip of the hair to dry the hair. In other words, controller 80 may particularly apply the cosmetic A effective for the root to the root of the hair, and particularly apply the cosmetic B effective for the tip to the tip of the hair.

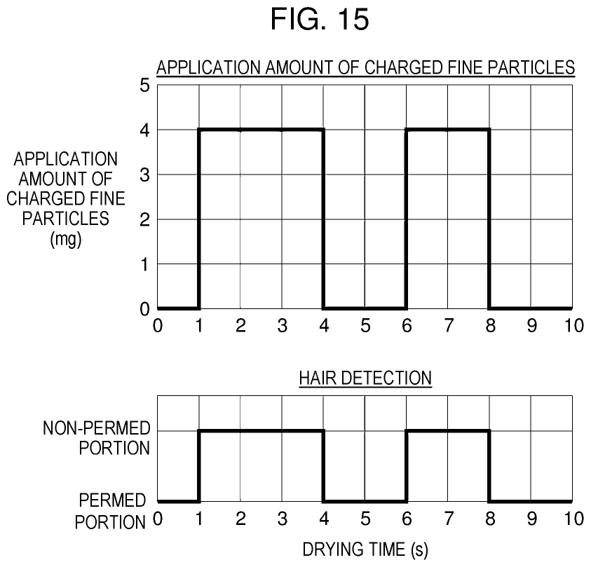

FIG. 15 is a timing chart illustrating an example of a relationship between the application amount of the charged fine particles and detection of a permed portion, which is adopted in a case where a user's hairstyle is a partial perm. The upper diagram shows the application amount (mg) of the charged fine particles relative to the drying time (seconds). Here, as an example, the application amount when charged fine particle water is applied is constant at 4 mg. The lower diagram shows whether the hair is a permed portion or a non-permed portion as a result of hair detection relative to a drying time (seconds). In FIG. 15, the drying time, being a horizontal axe in the upper diagram and the lower diagram, corresponds to each other. Controller 80 determines whether a part of the hair to be blown is a permed portion or a non-permed portion based on, for example, the output signal of wetting detection unit 60. As shown in FIG. 15, controller 80 may cause component generation unit 40 to apply the charged fine particle water only when determining that the part of the hair to be blown is a non-permed portion. As a result, hair dryer 1 can suppress elongation of the permed portion caused by moisture in advance.

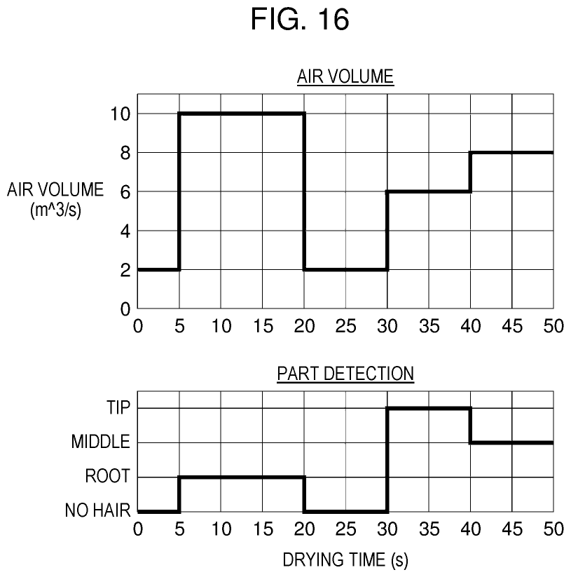

FIG. 16 is a timing chart illustrating an example of a relationship between an air volume and part detection of the user's hair. The upper diagram shows the air volume (m³/s) relative to the drying time (seconds). The lower diagram shows part detection of the hair relative to the drying time (seconds). In FIG. 16, the drying times, which are the horizontal axe in the upper diagram and the lower diagram, correspond to each other. Controller 80 determines a part that is blown by air from hair dryer 1 based on, for example, the output signal of part detection unit 64. Here, as shown in FIG. 16, controller 80 causes heat application unit 30 to blow air at an air volume of, for example, 2 (m³/s) while determining that there is no hair. On the other hand, controller 80 causes heat application unit 30 to blow air at an air volume of, for example, 10 (m³/s), while air is blown against the roots of the hair to dry the hair. Further, controller 80 causes heat application unit 30 to blow air at an air volume of, for example, 8 (m³/s) while air is blown against the middle of the hair to dry the hair. Furthermore, controller 80 causes heat application unit 30 to blow air at an air volume of, for example, 6 (m³/s), while air is blown against the tip of the hair to dry the hair. In other words, controller 80 may increase the air volume toward the root side of the hair and decrease the air volume toward the tip side of the hair. Note that, controller 80 may cause heat application unit 30 not to blow air while determining that there is no hair.

Next, an input screen on which a user inputs information and an output screen that displays information to the user will be described. In the present exemplary embodiment, display 73 is installed in housing 3 of main body 10. Therefore, an input screen and an output screen may be displayed on display 73. On the other hand, in a case where hair dryer 1 includes transmitting and receiving unit 74 that performs transmission and reception of various types of information with portable terminal device 100, an input screen and an output screen may be displayed on terminal display unit 101 of portable terminal device 100 instead of display 73. In other words, as long as hair dryer 1 includes transmitting and receiving unit 74, display 73 may not be included. In the following description, a case where an input screen and an output screen are displayed on terminal display unit 101 of portable terminal device 100 will be illustrated.

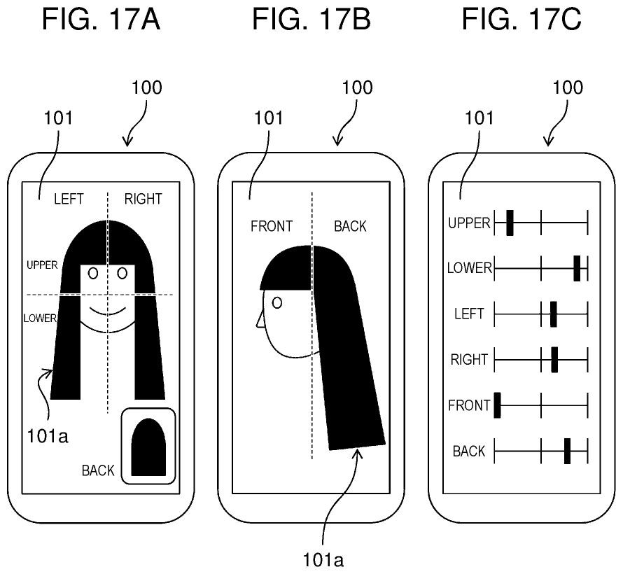

FIGS. 17A to 17C are schematic views illustrating a first example of an input screen displayed on terminal display unit 101 (or display 73). FIG. 17A shows a first input screen according to the first example. Image 101a displayed on the first input screen is a schematic view of the user's front hairstyle, and is a divided image divided into upper, lower, left, and right. On the first input screen, a schematic drawing of the user's rear hairstyle is also displayed. FIG. 17B shows a second input screen of the first example. Image 101a displayed on the second input screen is a schematic view of the user's side hairstyle, and is a divided image divided into front and rear. FIG. 17C shows a third input screen of the first example. On the third input screen, a level adjustment screen for the user to change the setting of a certain item is displayed for each area of the divided screens displayed on the first input screen and the second input screen.

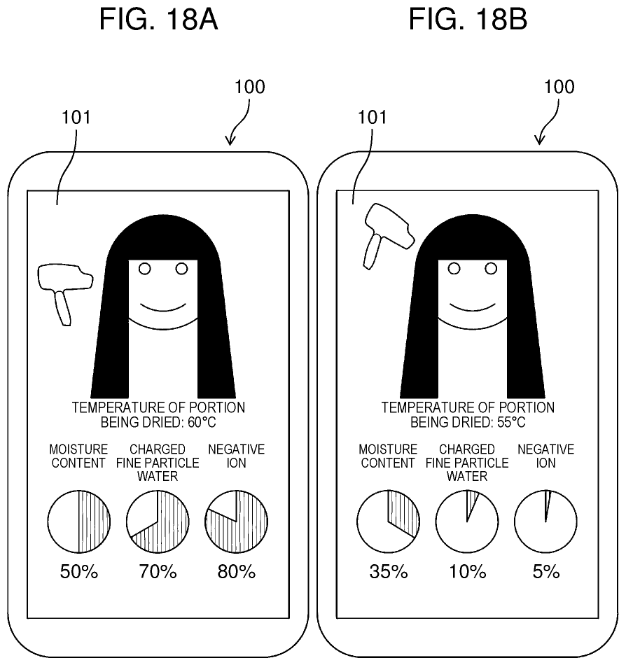

FIGS. 18A and 18B are schematic views illustrating a first example of an output screen displayed on terminal display unit 101 (or display 73). The output screen of the first example displays various states in real time related to a period in which hair dryer 1 is drying hair or a period in which hair dryer 1 is applying a component to hair. FIG. 18A is an output screen at the time when the middle of the hair is dried. FIG. 18B is an output screen at the time when the root of the hair is dried. Display items on the output screen of the first example are, for example, a part of the hair being dried, a temperature of the part being dried (a temperature of a portion being dried), the moisture content in the hair, and the amount of the component being applied, respectively, at the present time. In this example, the component amounts of charged fine particle water and negative ions are displayed as the amount of a component. As shown in FIGS. 18A and 18B, the part of the hair being dried may be visually indicated to the user by displaying a schematic drawing of a hair dryer relative to the schematic drawing of the hair. Similarly, the moisture content and the amount of a component may be visually indicated to the user by displaying a pie chart, for example, in addition to numerical values.

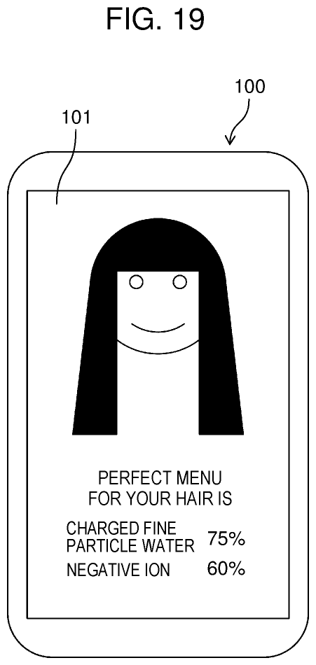

FIG. 19 is a schematic view illustrating a second example of the output screen displayed on terminal display unit 101 (or display 73). The output screen of the second example displays various states in non-real time after hair dryer 1 dries at least a part of the hair or applies a component to at least a part of the hair. As shown in FIG. 19, as a result of using hair dryer 1 by the user, the amount of an effective component for the user's hair may be displayed. In this example, the component amounts of charged fine particle water and negative ions are displayed.

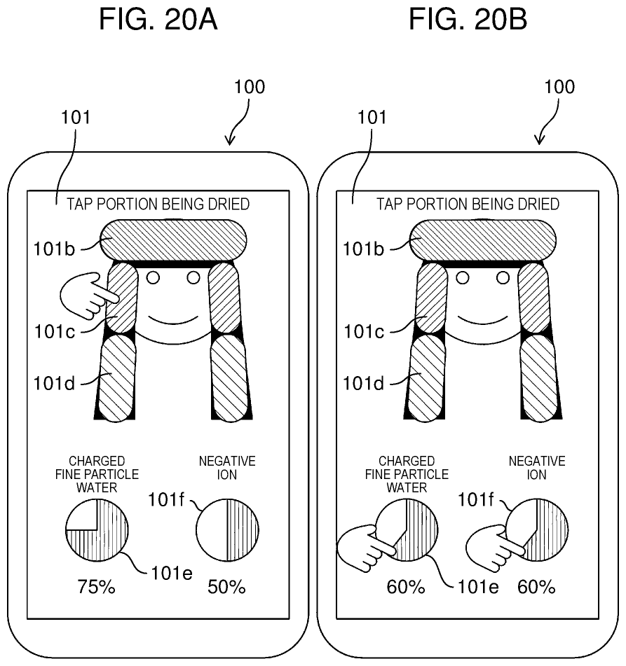

FIGS. 20A and 20B are schematic views illustrating a third example of the output screen displayed on terminal display unit 101 (or display 73). The output screen of the third example displays various states in non-real time, similarly to the output screen of the second example. Further, the output screen of the third example displays the component amount for each part of the user's hair, and displays the part of the hair and the component amount for each part such that the user can change them. First, on the output screen of the third example, a schematic drawing of the user's hair with three types of tap areas is displayed. First tap area 101b corresponds to a part at the root of the hair. Second tap area 101c corresponds to a part at the middle of the hair. Third tap area 101d corresponds to a part at the tip of the hair. Further, on the output screen of the third example, the component amount at the present time is displayed in a pie chart, for example, in addition to numerical values. For example, first pie chart 101e indicates the component amount of charged fine particle water. Second pie chart 101f indicates the component amount of negative ions. FIG. 20A shows, as an example, a state in which the user selects the middle as the part of the hair for which the component amount is desired to be changed by the subsequent operation of hair dryer 1. In this case, the user can select the middle by tapping second tap area 101c on the third output screen. FIG. 20B shows, as an example, a state in which the user inputs a component amount to be newly set when the user wants to change the component amount by the subsequent operation of hair dryer 1. The user can set a desired component amount by changing the display value while tapping first pie chart 101e and second pie chart 101f on the third output screen.

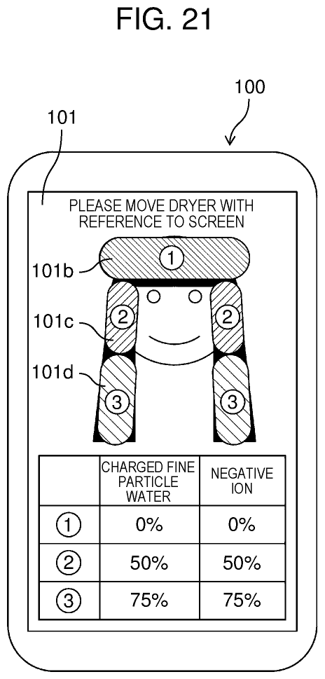

FIG. 21 is a schematic view illustrating a fourth example of the output screen displayed on terminal display unit 101 (or display 73). The output screen of the fourth example displays various states in non-real time, similarly to the output screen of the third example. In the output screen of the third example shown in FIG. 20A, when any one of first tap area 101b, second tap area 101c, and third tap area 101d is tapped, the component amount in the part corresponding to the area is displayed. On the other hand, on the output screen of the fourth example, the component amount for each part of the hair are collectively displayed, and the order of the parts to which the user should apply heat or a component by hair dryer 1 is displayed with numerals. The user can efficiently apply a desired component by moving hair dryer 1 so as to apply heat or a component in the order of the numbers attached to each of first tap area 101b, second tap area 101c, and third tap area 101d.

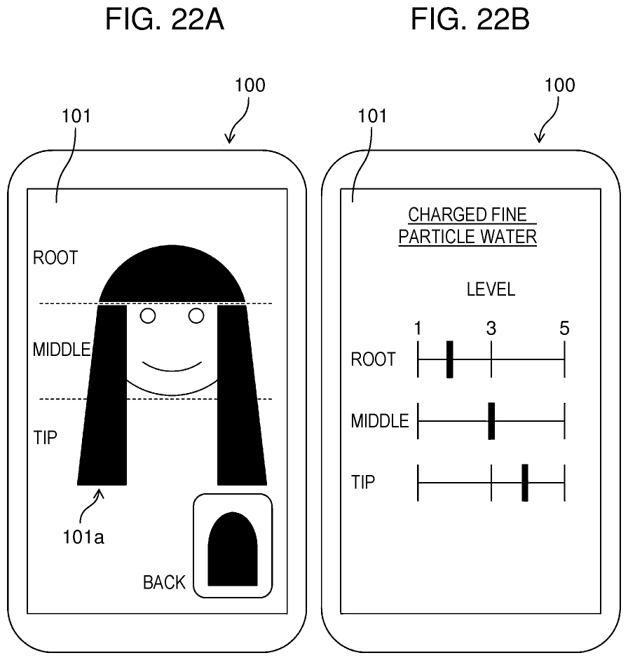

FIGS. 22A and 22B are schematic views illustrating a second example of the input screen displayed on terminal display unit 101 (or display 73). The input screen of the second example corresponds to a case where the user changes the component to be applied to each part of the hair. FIG. 22A shows a first input screen of the second example. Image 101a displayed on the first input screen is a schematic view of the user's front hairstyle, and is a divided image divided into three parts of the root, the middle, and the tip in the vertical direction. On the first input screen, a schematic drawing of the user's rear hairstyle is also displayed. FIG. 22B shows a second input screen of the second example. On the second input screen, a level adjustment screen for the user to change the setting of charged fine particle water is displayed for each area of the divided screen displayed on the first input screen. In a case where the user wants to change charged fine particle water to his or her desired component amount instead of the component amount set by controller 80, first, on the first input screen, three parts in which the amount of the component can be changed in the hair are presented as image 101a. Next, the user can display the second input screen and change the component amount of the charged fine particle water at a plurality of levels so that the component amount of the charged fine water particles becomes a desired component amount for each part.

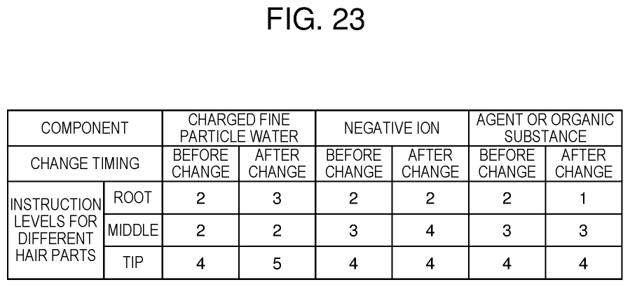

FIG. 23 is a table illustrating a setting example in a case where the component amount is changed for each part of the hair using the input screen of the second example shown in FIG. 22B. In a case where the component to be changed is charged fine particle water as illustrated in FIG. 22B, for example, when the level of the charged fine particle water applied to the root of the hair before the change is “2,” the user may increase the level to “3” by using the input screen of the second example according to his or her preference. The user can change the component amount of the charged fine particle water to a desired component amount by changing the level in the same way in the middle or the tip of the hair, which are other parts of the hair. Further, the user can change the component amount of not only the charged fine particle water but also other components such as negative ions, an agent, and an organic matter, using the input screen of the second example in the same way.

Next, how controller 80 estimates the degree of dryness of the hair when the user's hair is being dried will be described.

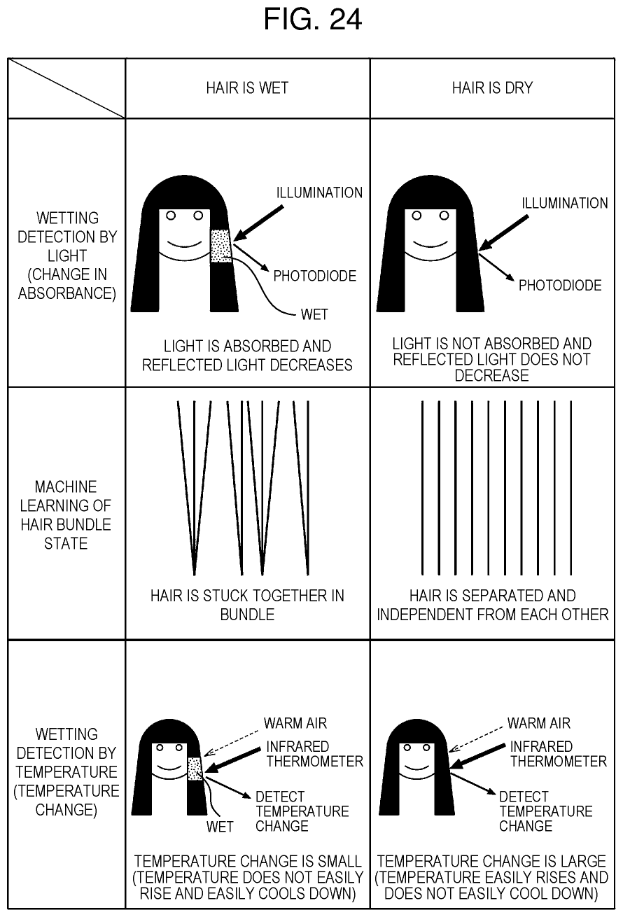

FIG. 24 is a table illustrating several principles that can be used to determine whether hair is wet or dry. To start with, in the present exemplary embodiment, the degree of dryness of the hair is estimated by drying estimation calculation unit 87 based on the wetting information calculated by wetting calculation unit 86. Further, in the present exemplary embodiment, wetting detection unit 60 is specifically wetting detection sensor 60a that is a photodiode. The wetting information is the absorbance calculated by wetting calculation unit 86 based on the signal intensity from wetting detection sensor 60a. As shown in the upper column in FIG. 24, in a case where the hair is wet, a large amount of light is absorbed by the hair when the hair is irradiated with light from illumination unit 72, so that reflected light received by wetting detection sensor 60a is decreased. On the other hand, in a case where the hair is dry, the amount of light absorbed by the hair is small when the hair is irradiated with light from illumination unit 72, so that the reflected light received by wetting detection sensor 60a does not decrease. That is, drying estimation calculation unit 87 can estimate the degree of dryness, in other words, whether the hair is wet or dry, based on the change in absorbance.

Further, as another principle, the degree of dryness of the hair may be calculated by machine learning using a bundle state of the hair as wetting information. In this case, wetting detection unit 60 is a photographing unit such as a camera that photographs hair. Wetting calculation unit 86 is a machine learning calculation unit that discriminates the bundle state of the hair based on a hair image photographed by wetting detection unit 60. As shown in the middle column in FIG. 24, in a case where the hair is wet, the hair is stuck together and bundled. On the other hand, in a case where the hair is dry, the hair is separated and independent from each other. In other words, drying estimation calculation unit 87 can estimate the degree of dryness based on the bundle state of the hair discriminated by machine learning calculation unit.

Furthermore, the degree of dryness of the hair may be calculated by wetting calculation unit 86 using the temperature of the hair as wetting information. In this case, wetting detection unit 60 is a temperature sensor. The temperature sensor may be, for example, an infrared thermometer (an infrared sensor). Wetting calculation unit 86 calculates a temperature as wetting information based on the hair measurement value measured by wetting detection unit 60. As shown in the lower column of FIG. 24, in a case the hair is wet, when warm air is sent from discharge port 10b toward the hair, the temperature of the surface of the hair is less likely to rise and easily cools down, so that a temperature change is small. On the other hand, in a case where the hair is dry, when warm air is sent from discharge port 10b toward the hair, the temperature of the surface of the hair easily rises and is less likely to cool down, so that the temperature change is large. In other words, drying estimation calculation unit 87 can estimate the degree of dryness based on the change in the temperature of the hair.

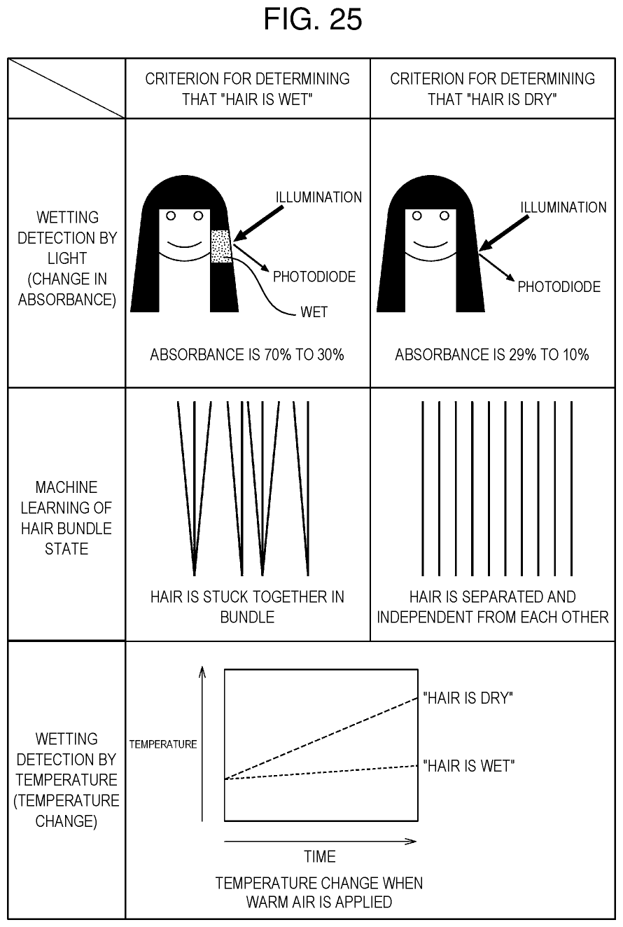

FIG. 25 is a table illustrating specific criteria for determining whether hair is wet or dry, which correspond to what is shown in FIG. 24. To start with, when a determination is made based on the change in absorbance as in the present exemplary embodiment, as shown in the upper column in FIG. 25, it may be determined that the hair is wet when the absorbance is 70% to 30%, while it may be determined that the hair is dry when the absorbance is 29% to 10%. Further, when the determination is made based on the bundle state of the hair, the determination may be made according to the result of the machine learning as shown in the middle columns in FIGS. 24 and 25. Furthermore, when the determination is made based on the change in temperature, as shown in the lower column in FIG. 25, it may be determined that the hair is wet when a gradient of the change in temperature when warm air is blown against the hair is gentle, and it may be determined that the hair is dry when the gradient of the change in temperature is steep.

Next, effects of hair dryer 1 will be described.

Hair dryer 1 as a hair care device according to the present exemplary embodiment includes heat application unit 30 that applies heat to a user's hair, component generation unit 40 that generates a component acting on the hair, and part detection unit 64 that detects at least a part of the hair. Further, hair dryer 1 also includes controller 80 that estimates a part to which heat or the component is applied based on an output of part detection unit 64, and adjusts, for each part, amount of heat set in heat application unit 30 or a component amount set in component generation unit 40.

In the present exemplary embodiment, when heat or a component is applied to the user's hair, controller 80 estimates a part to which heat is applied from heat application unit 30 or to which the component is applied from component generation unit 40. Then, controller 80 adjusts the amount of heat and the component amount to be applied to each estimated part of the user's hair, so that fine-grained control optimal for the user using hair dryer 1 can be executed.

As described above, according to the present exemplary embodiment, it is possible to provide a hair care device that easily leads to a finish of hair desired by a user.

Further, in hair dryer 1, controller 80 includes part calculation unit 91 that estimates a part based on the output from part detection unit 64. Further, controller 80 includes accumulative calculation unit 88 that calculates, for each part estimated by part calculation unit 91, an accumulative heat amount that is an accumulative amount of heat applied by heat application unit 30 or an accumulative component amount that is an accumulative amount of the component applied by component generation unit 40. The amount of heat may be adjusted with reference to the accumulative heat amount. The component amount may be adjusted with reference to the accumulative component amount.

According to such hair dryer 1, the amount of heat is adjusted with reference to the accumulative heat amount, or the component amount is adjusted with reference to the accumulative component amount, and thus controller 80 can adjust the amount of heat or the component amount to be applied to each part of the hair by simple control.

Further, in hair dryer 1, part detection unit 64 may be at least one of orientation detection unit 64b with at least one axis, distance measurement unit 64a, and surface condition detection unit 64c. Here, orientation detection unit 64b detects a position or an orientation of hair dryer 1 as a hair care device. Distance measurement unit 64a measures a distance to the hair or the user's skin. Surface condition detection unit 64c detects a surface condition of the hair.

According to such hair dryer 1, with a simpler configuration, part detection unit 64 can detect a part to which heat is applied by heat application unit 30 or a part to which a component is applied by component generation unit 40. Alternatively, controller 80 can cause part calculation unit 91 to estimate a part to which heat is applied by heat application unit 30 or a part to which a component is applied by component generation unit 40 by simpler control.

Further, in hair dryer 1, when part detection unit 64 is orientation detection unit 64b, part calculation unit 91 obtains a variation amount relative to an initial position set in advance with respect to the output of orientation detection unit 64b for a predetermined time. Here, part calculation unit 91 may determine that the user is performing a drying operation in a case where the variation amount is smaller than a predefined reference amount. On the other hand, part calculation unit 91 may determine that the user is not performing the drying operation in a case where the variation amount is larger than the predetermined reference amount.

According to such hair dryer 1, when part detection unit 64 is orientation detection unit 64b, part calculation unit 91 can easily determine whether the user is performing the drying operation.

Further, in hair dryer 1, when part detection unit 64 is distance measurement unit 64a or surface condition detection unit 64c, part calculation unit 91 obtains the variation amount of the output of distance measurement unit 64a or surface condition detection unit 64c for a predetermined time. Part calculation unit 91 may determine the part based on the magnitude of the variation amount.

According to such hair dryer 1, part calculation unit 91 can easily determine apart to which heat is applied by heat application unit 30 or a part to which a component is applied by component generation unit 40. Further, accumulative calculation unit 88 can reduce an error in the component application amount due to hair fluttering, for example, by referring to the part determined by part calculation unit 91 during calculation.

Further, hair dryer 1 includes measurement unit 50 that measures or photographs hair. Controller 80 includes drying estimation calculation unit 87 that estimates the degree of dryness of the hair based on the hair measurement value or the hair image obtained from measurement unit 50. Accumulative calculation unit 88 may correct the accumulative heat amount or the accumulative component amount based on the degree of dryness estimated by drying estimation calculation unit 87.

According to such hair dryer 1, during the drying operation, controller 80 adjusts the accumulative heat amount and the accumulative component amount with reference to the degree of dryness of the hair, so that the component adjusted to a more optimal component amount can be applied to the hair.

Further, hair dryer 1 also includes display 73 that displays at least a divided image divided into at least two in a front-back direction, a left-right direction, or an up-down direction of hair. Controller 80 may change the component amount to a desired amount based on a divided portion selected by the user in the divided image on display 73.

According to such hair dryer 1, the component amount set by controller 80 can be changed to the component amount desired by the user using a split screen, so that it can be easier to lead to a finish of hair desired by the user.

Further, hair dryer 1 also includes transmitting and receiving unit 74 that performs transmission and reception with terminal communication unit 103 provided in portable terminal device 100 as an external communication device. Here, assume that at least a divided image divided into at least two of a front-back direction, a left-right direction, or an up-down direction of hair are displayed on terminal display unit 101 provided in portable terminal device 100. At this time, transmitting and receiving unit 74 may receive information related to the divided portion selected by the user in the divided image of terminal display unit 101 from terminal communication unit 103. Further, controller 80 may change the component amount to a user's desired amount based on the information related to the divided portion received by transmitting and receiving unit 74 from terminal communication unit 103.

With such hair dryer 1, the user can adjust the settings in hair dryer 1 from portable terminal device 100, so that convenience for the user can be improved.

### Second Exemplary Embodiment

Hair dryer 1 according to the first exemplary embodiment described above adopts wetting detection sensor 60a (a photodiode) as an example of wetting detection unit 60. On the other hand, in hair dryer 1 according to the second exemplary embodiment, as an example of wetting detection unit 60, photographing unit 60b is adopted instead of wetting detection sensor 60a.

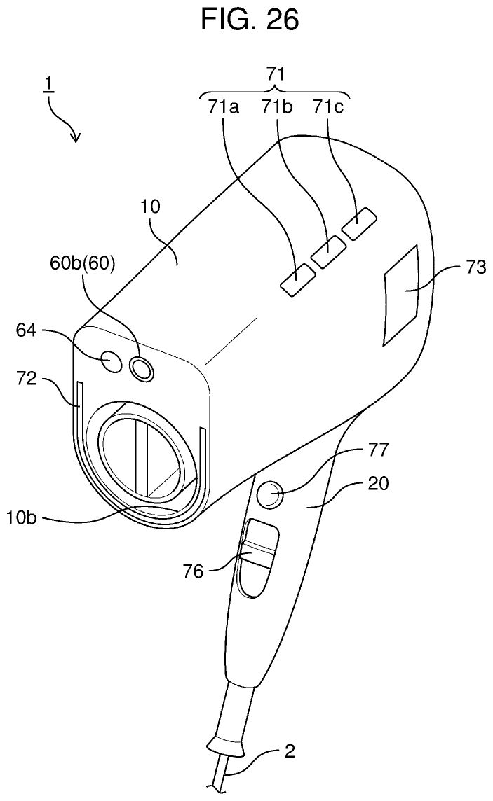

FIG. 26 is a schematic perspective view illustrating a configuration of hair dryer 1 as a hair care device according to the second exemplary embodiment. Hair dryer 1 according to the present exemplary embodiment includes photographing unit 60b installed instead of wetting detection sensor 60a in the first exemplary embodiment, and illumination unit 72 installed so as to surround a part of discharge port 10b. Note that, in hair dryer 1 described herein, since the configuration other than photographing unit 60b and illumination unit 72 is the same as the configuration in the first exemplary embodiment (excluding the configuration related to control of, for example, controller 80 and signal processing unit 90), the same reference numerals are given, and a detailed description thereof is omitted.

To start with, in a case where photographing unit 60b is used as wetting detection unit 60, as described above with reference to FIGS. 24 and 25, drying estimation calculation unit 87 can estimate the degree of dryness by machine learning based on a hair image. In this case, a point expressed as a “hair measurement value” in the description of hair dryer 1 according to the first exemplary embodiment can be replaced with a “hair image” in the present exemplary embodiment.

Further, in the case where photographing unit 60b is used as wetting detection unit 60, hair dryer 1 may include part detection unit 64 as illustrated in the first exemplary embodiment separately from photographing unit 60b. In other words, hair dryer 1 according to the present exemplary embodiment has the same advantageous effects as hair dryer 1 according to the first exemplary embodiment.

### Other Exemplary Embodiments

A hair care device according to another exemplary embodiment of the present disclosure may include a part detection unit, an accumulative amount calculation unit, and a hair application control unit. The part detection unit detects a part to which a component or heat is applied to a user's hair. The accumulative amount calculation unit calculates the component or heat accumulated in the whole or a part of the hair by using detection information detected by the part detection unit. The hair application control unit generates and controls the component or heat using estimation data calculated by the accumulative amount calculation unit, and applies the component or heat to the hair. Here, the part detection unit may be, for example, an alternative to part detection unit 64 in each of the exemplary embodiments described above. The accumulative amount calculation unit may be, for example, an alternative to accumulative calculation unit 88 in each of the exemplary embodiments described above. Further, the hair application control unit may be, for example, an alternative to at least one of heat application unit 30, component generation unit 40, application amount calculation unit 83, component amount control unit 84, and heat amount control unit 85 in each of the exemplary embodiments described above.

The hair application control unit may control a component amount of the component or an increase or decrease in heat.

The part detection unit may include a hair wetting detection unit that detects a wetting state of the whole or part of the user's hair. The hair wetting detection unit may be, for example, an alternative to wetting detection unit 60 in each of the exemplary embodiments described above.

The accumulative amount calculation unit may include a hair drying estimation calculation unit that estimates the degree of dryness of the hair using hair wetting detection data detected by the hair wetting detection unit. The hair drying estimation calculation unit may be, for example, an alternative to drying estimation calculation unit 87 in each of the exemplary embodiments described above.

Further, the accumulative amount calculation unit may correct the estimation data using hair dryness data estimated by the hair drying estimation calculation unit.

The hair application control unit may include a screen capable of inputting and outputting data for controlling the hair care device. The screen herein may be, for example, an alternative to display 73 in each of the exemplary embodiments described above.

On the other hand, the screen included in the hair application control unit may be provided separately from the hair care device. The screen herein may be, for example, an alternative to terminal display unit 101 included in portable terminal device 100 in each of the exemplary embodiments described above.

Further, the screen included in the hair application control unit may display a divided view divided into at least two in a front-back direction, a left-right direction, or an up-down direction of the hair, and control the hair care device for each divided view. The divided view may be an alternative to image 101a as a divided image as described with reference to FIG. 17A or the like in the first exemplary embodiment.

Further, the hair care device according to the present exemplary embodiment may include a data transmission and reception unit for performing transmission and reception with outside of the hair care device. The data transmission and reception unit may be, for example, an alternative to transmitting and receiving unit 74 in each of the exemplary embodiments described above.

Furthermore, the hair application control unit may perform intermittent operation control when applying the component or heat to the user's hair. Here, the intermittent operation control refers to, for example, the control as described using the timing charts in FIGS. 11 to 16 in the first exemplary embodiment.

Note that, since the exemplary embodiments described above are intended to illustrate the technique in the present disclosure, various changes, replacements, additions, omissions, and the like may be made within the scope of the claims or equivalents thereof.

### INDUSTRIAL APPLICABILITY

The present disclosure is applicable to all hair care devices for home use or business use that dry a user's hair or adjust a hairstyle of a user.

### REFERENCE MARKS IN THE DRAWINGS

1 hair dryer
2 power supply cord
3 housing
3a partition plate
4 air blowing flow channel
10 main body
10a suction port
10b discharge port
10c connecting part
10d connecting shaft
10e branch channel
10f component discharge port
10g front surface portion
14 nozzle part
20 grip part
20a housing
30 heat application unit
31 fan
32 motor
33 heating unit
40 component generation unit
40a first electrostatic atomization device
40b second electrostatic atomization device
40c third electrostatic atomization device
41a mist atomizer
41b tank
41c pump
41d GND electrode
41e high voltage circuit
41f pump drive circuit
42a discharger
42b GND electrode
42c high voltage circuit
43a discharger
43b Peltier element
43c GND electrode
43d high voltage circuit
50 measurement unit
60 wetting detection unit
60a wetting detection sensor
60b photographing unit
61 room temperature sensor
62 humidity sensor
63 hair detection unit
64 part detection unit
64a distance measurement unit
64b orientation detection unit
64c surface condition detection unit
71 input unit
71a hair quality input unit
71b hair length input unit
71c hair volume input unit
72 illumination unit
73 display
74 transmitting and receiving unit
75 storage
76 power supply switch
77 initialization switch
80 controller
81 hair characteristic recognition unit
82 table generation unit
83 application amount calculation unit
84 component amount control unit
85 heat amount control unit
86 wetting calculation unit
87 drying estimation calculation unit
88 accumulative calculation unit
90 signal processing unit
91 part calculation unit
92 initial position determination unit
100 portable terminal device
101 terminal display unit
101a image
101b first tap area
101c second tap area
101d third tap area
101e first pie chart
101f second pie chart
102 terminal photographing unit
103 terminal communication unit

1. A hair care device comprising:
a heat application unit that applies heat to hair of a user;
a component generation unit that generate a component acting on the hair;
a part detection unit that detects at least a part of the hair; and
a controller that estimates a part to which the heat or the component is applied based on an output of the part detection unit, and adjusts amount of heat set in the heat application unit or a component amount set in the component generation unit for each part.

2. The hair care device according to claim 1, wherein
the controller includes
a part calculation unit that estimates the part based on an output from the part detection unit, and
an accumulative calculation unit that calculates, for each part estimated by the part calculation unit, an accumulative heat amount that is an accumulative amount of the heat applied by the heat application unit or an accumulative component amount that is an accumulative amount of the component applied by the component generation unit,
the amount of heat is adjusted with reference to the accumulative heat amount, and
the component amount is adjusted with reference to the accumulative component amount.

3. The hair care device according to claim 2, wherein the part detection unit is at least one of an orientation detection unit with at least one axis that detects a position or an orientation of the hair care device, a distance measurement unit that measures a distance to the hair or skin of the user, and a surface condition detection unit that detects a surface condition of the hair.

4. The hair care device according to claim 3, wherein when the part detection unit is the orientation detection unit, the part calculation unit obtains a variation amount relative to an initial position set in advance with respect to the output of the orientation detection unit for a predetermined time, determines that the user is performing a drying operation when the variation amount is smaller than a predefined reference amount, and determines that the user is not performing the drying operation when the variation amount is larger than the reference amount.

5. The hair care device according to claim 3, wherein when the part detection unit is the distance measurement unit or the surface condition detection unit, the part calculation unit obtains a variation amount of the output of the distance measurement unit or the surface condition detection unit for a predetermined time, and determines the part based on a magnitude of the variation amount.

6. The hair care device according to claim 2, comprising a measurement unit that measures or photographs the hair,
wherein
the controller includes a drying estimation calculation unit that estimates a degree of dryness of the hair based on a hair measurement value or a hair image obtained from the measurement unit, and
the accumulative calculation unit corrects the accumulative heat amount or the accumulative component amount based on the degree of dryness estimated by the drying estimation calculation unit.

7. The hair care device according to any one of claims 1 to 6, comprising a display that displays at least a divided image divided into at least two in a front-back direction, a left-right direction, or an up-down direction of the hair,
wherein the controller changes the component amount to a desired amount based on a divided portion selected by the user in the divided image on the display.

8. The hair care device according to any one of claims 1 to 6, comprising a transmitting and receiving unit that performs transmission and reception with a terminal communication unit included in a portable terminal device as an external communication device,
wherein
when at least a divided image divided into at least two in a front-back direction, a left-right direction, or an up-down direction of the hair is displayed on a terminal display unit provided in the portable terminal device,
the transmitting and receiving unit receives, from the terminal communication unit, information on a divided portion selected by the user in the divided image on the terminal display unit, and
the controller changes the component amount to an amount desired by the user based on the information on the divided portion received by the transmitting and receiving unit from the terminal communication unit.

9. A hair care device comprising:
a part detection unit that detects a part to which a component or heat is applied to hair of a user;
an accumulative amount calculation unit that calculates the component or the heat accumulated in a whole or a part of the hair by using detection information detected by the part detection unit; and
a hair application control unit that generates and controls the component or the heat using estimation data calculated by the accumulative amount calculation unit and applies the component or the heat to the hair.

10. The hair care device according to claim 9, wherein the hair application control unit controls a component amount of the component or an increase or decrease in the heat.

11. The hair care device according to claim 9, wherein the part detection unit includes a hair wetting detection unit that detects a wetting state of the whole or part of the hair of the user.

12. The hair care device according to claim 11, wherein the accumulative amount calculation unit includes a hair drying estimation calculation unit that estimates a degree of dryness of the hair using hair wetting detection data detected by the hair wetting detection unit.

13. The hair care device according to claim 12, wherein the accumulative amount calculation unit corrects the estimation data using hair dryness data estimated by the hair drying estimation calculation unit.

14. The hair care device according to claim 9, wherein the hair application control unit includes a screen capable of inputting and outputting data for controlling the hair care device.

15. The hair care device according to claim 14, wherein the screen is provided separately from the hair care device.

16. The hair care device according to claim 14, wherein the screen displays a divided view divided into at least two in a front-back direction, a left-right direction, or an up-down direction of the hair, and controls the hair care device for each divided view.

17. The hair care device according to claim 9, comprising a data transmission and reception unit that performs transmission and reception with outside of the hair care device.

18. The hair care device according to claim 9, wherein the hair application control unit performs intermittent operation control when applying the component or the heat to the hair of the user.
